(19) United States
(12) Patent Application Publication
(10) Pub. No.: US 2026/0139238 A1
(43) Pub. Date: May 21, 2026

(54) METHODS AND PRODUCTS FOR REMOVAL OF URACIL CONTAINING POLYNUCLEOTIDES

(71) Applicant: DNA SCRIPT, Le Kremlin-Bicêtre (FR)

(72) Inventors: Mikhael SOSKINE, Le Kremlin-Bicêtre (FR); Puay Suan Jasmine CHUA, Le Kremlin-Bicêtre (FR); Wesley LOFTIE-EATON, Le Kremlin-Bicêtre (FR); Elise CHAMPION, Le Kremlin-Bicêtre (FR)

(21) Appl. No.: 19/122,511

(22) PCT Filed: Oct. 18, 2023
(86) PCT No.: PCT/EP2023/078926

§ 371 Date: Apr. 18, 2025

(30) Foreign Application Priority Data
Oct. 19, 2022 (EP) 22202582.7

(51) Int. Cl. C12N 9/24; C12P 19/34

(52) U.S. Cl. CPC C12N 9/2497; C12P 19/34; C07K 2319/20; C12Y 302/02027

(57) ABSTRACT

The present invention relates to the use of a DNA glycosylase enzyme for removing or blocking uracil containing polynucleotides, and methods incorporating the use of such enzymes to remove or block uracil containing poly nucleotides from polynucleotide containing compositions, particularly in methods of template-free DNA synthesis The present invention further relates to methods of tethering polynucleotides to a surface or molecule using a DNA glycosylase enzyme, the resulting products therefrom, and to modified DNA glycosylase enzymes.

### FIELD OF THE INVENTION

The present invention relates to the use of a DNA glycosylase enzyme for removing or blocking uracil containing polynucleotides, and methods incorporating the use of such enzymes to remove or block uracil containing polynucleotides from polynucleotide containing compositions, particularly in methods of template-free polynucleotide synthesis. The present invention further relates to methods of tethering polynucleotides to a surface or molecule using a DNA glycosylase enzyme, the resulting products therefrom, and to modified DNA glycosylase enzymes.

### Introduction to the Invention

Interest in enzymatic approaches to polynucleotide synthesis has recently increased both because of increased demand for synthetic polynucleotides that are made to order for use in many areas of biotechnology such as CRISPR-Cas9 applications, high-throughput sequencing, labelling, PCR, and the like, and also due to the limitations of chemical approaches to polynucleotide synthesis, as described in Jensen et al. Biochemistry, 57:1821-1832 (2018).

Currently, most enzymatic synthesis approaches employ a template-free polymerase to repeatedly add blocked or protected nucleoside triphosphates to the free end of an initiator polynucleotide or subsequently elongated polynucleotide attached to a solid support, followed by rounds of deblocking and polymerisation until the desired polynucleotide is obtained. These methods are highly effective, however the deblocking step can cause issues with base transitions.

It is a common problem in enzymatic synthesis processes that the deblocking chemistry can generate cytosine to thymine transitions during the polymerisation, which is believed to be caused by the deamination of cytosine bases. This leads to the subsequent formation of uracil bases which contaminate the polynucleotide product and generate incorrect sequence mutations. This is undesirable because it lowers the purity of the final product.

Some techniques have been developed in the art to address this problem. These include removing the uracil base by cleaving the polynucleotide immediately 5′ of the base using an endonuclease enzyme which is specific for uracil bases. However, this technique relies on destroying the uracil containing polynucleotides rather than simply removing them. Another possibility is to use antibodies specific for uracil which bind to the polynucleotide and allow its removal. But this is cumbersome and involves expensive reagents.

It would be an improvement to current enzymatic polynucleotide synthesis processes if a way of removing the uracil-containing polynucleotide contaminates could be developed which is simple and effective.

It is the aim of one or more aspects of the present invention to solve one or more of the above-mentioned problems in the art.

### SUMMARY OF THE INVENTION

According to an aspect of the present invention there is provided the use of a uracil DNA glycosylase enzyme for removing a uracil containing polynucleotide from a composition containing at least one polynucleotide, wherein the uracil DNA glycosylase enzyme is capable of forming a stable bond with the uracil containing polynucleotide.

According to another aspect of the present invention there is provided the use of a uracil DNA glycosylase enzyme for blocking a uracil containing polynucleotide within a composition containing at least one polynucleotide, wherein the uracil DNA glycosylase enzyme is capable of forming a stable bond with the uracil containing polynucleotide.

According to another aspect of the present invention there is provided the use of a uracil DNA glycosylase enzyme in a method of polynucleotide synthesis, wherein the uracil DNA glycosylase enzyme is capable of forming a stable bond with a uracil containing polynucleotide.

According to another aspect of the present invention there is provided the use of a uracil DNA glycosylase enzyme for tethering a uracil containing polynucleotide to a surface or to a molecule, wherein the uracil DNA glycosylase enzyme is capable of forming a stable bond with a uracil containing polynucleotide.

According to another aspect of the present invention there is provided the use of a uracil DNA glycosylase enzyme for error correction of C to U deamination error. In DNA, spontaneous cytosine into uracil can happen and needs to be corrected. According to another aspect of the present invention there is provided the use of a uracil DNA glycosylase enzyme for fluorescent labelling of uracil-containing DNA via a pre-labelled DNA glycosylase.

According to another aspect of the present invention there is provided the use of uracil DNA glycosylase to tether an enzyme, such as TdT, to initiator nucleic acid for the purpose of DNA synthesis. In a particular aspect, the initiator nucleic acid is immobilized on the surface.

According to another aspect of the present invention there is provided a method of removing at least one uracil containing polynucleotide from a composition containing at least one polynucleotide, the method comprising the steps of:
(a) Providing a composition containing at least one polynucleotide;
(b) Contacting the composition with at least one uracil DNA glycosylase enzyme, wherein each enzyme is capable of forming a stable bond with a uracil containing polynucleotide, to form one or more enzyme-uracil containing polynucleotide complexes;
(c) Separating the one or more enzyme-uracil containing polynucleotide complexes from the composition.

According to another aspect of the present invention there is provided a method of blocking at least one uracil containing polynucleotide within a composition containing at least one polynucleotide, the method comprising:
(a) Providing a composition containing at least one polynucleotide;
(b) Contacting the composition with at least one uracil DNA glycosylase enzyme, wherein each enzyme capable of forming a stable bond with a uracil containing polynucleotide, to form one or more enzyme-uracil containing polynucleotide complexes.

According to another aspect of the present invention there is provided a method of polynucleotide synthesis, the method comprising a step of: adding at least one uracil DNA glycosylase enzyme, wherein each enzyme capable of forming a stable bond with a uracil containing polynucleotide, to form one or more enzyme-uracil containing polynucleotide complexes.

According to another aspect of the present invention there is provided a method of tethering at least one polynucleotide to a surface, the method comprising the steps of:
(a) Depositing one or more uracil DNA glycosylase enzymes on a surface, wherein each uracil DNA glycosylase enzyme is capable of forming a stable bond with a uracil containing polynucleotide;
(b) Contacting the surface with at least one uracil containing polynucleotide to form one or more enzyme-uracil containing polynucleotide complexes.

According to another aspect of the present invention there is provided a method of tethering a polynucleotide to a molecule of interest, the method comprising the steps of:
(a) Attaching a uracil DNA glycosylase enzyme to a molecule of interest to form an enzyme-molecule of interest complex, wherein the uracil DNA glycosylase enzyme is capable of forming a stable bond with a uracil containing polynucleotide;
(b) Contacting the enzyme-molecule of interest complex with a uracil containing polynucleotide to allow the uracil DNA glycosylase enzyme to bind to the uracil containing polynucleotide.

According to another aspect of the present invention there is provided a composition containing at least one polynucleotide obtained from the method of any one of the fifth, sixth or seventh aspects.

According to another aspect of the present invention there is provided a surface comprising one or more uracil DNA glycosylase enzyme-uracil containing polynucleotide complexes tethered thereto.

According to another aspect of the present invention there is provided a complex comprising a uracil DNA glycosylase enzyme attached to a molecule of interest, wherein the uracil DNA glycosylase enzyme is further stably bound to a uracil containing polynucleotide.

According to another aspect of the present invention there is provided a uracil DNA glycosylase enzyme comprising an amino acid sequence having at least 70% identity with a sequence according to SEQ ID NO: 2, 7 or 9, and comprising one or more ancestral substitution mutations.

According to another aspect of the present invention there is provided a nucleic acid encoding the uracil DNA glycosylase enzyme of the thirteenth aspect.

According to another aspect of the present invention there is provided a host cell comprising the uracil DNA glycosylase enzyme of the thirteenth aspect or the nucleic acid of the fourteenth aspect.

According to another aspect of the present invention, there is provided a kit comprising a uracil DNA glycosylase enzyme of the thirteenth aspect, and one or more reagents for synthesis, amplification and/or sequencing of a polynucleotide.

The invention described herein is based on a particular type of uracil DNA glycosylase enzyme termed ‘UDGx’ which strongly binds to uracil without dissociating. The inventors have found that this enzyme can be used to improve methods of synthesising polynucleotides. Unlike typical uracil DNA glycosylase enzymes, which bind to uracil, excise the uracil base, and then dissociate, UDGx enzymes bind to uracil and excise the base but remain bound to the abasic site left behind. The enzyme therefore forms complexes with uracil containing polynucleotides. The inventors have realised and successfully applied UDGx enzymes to compositions containing a mixture of polynucleotides, such as those generated from enzymatic synthesis methods, to remove or block uracil containing polynucleotide contaminants. The inventors have found that the UDGx-uracil containing polynucleotide complexes which are formed are relatively inert and can either be removed from compositions containing polynucleotides using tag and capture mechanisms, or left within said compositions to block the uracil containing polynucleotides from any further processing steps such as sequencing. If the complexes are left within a composition, then the inventors have found that polymerisation machinery used in PCR or sequencing technologies is blocked from reading the uracil containing polynucleotide, effectively silencing the incorrect polynucleotides. ‘UDGx’ enzymes have never been applied in such a manner as to improve the quality of enzymatically synthesised polynucleotides.

Advantageously the inventors have further found that the irreversible nature of the bond between the UDGx enzyme and the uracil containing polynucleotide means that the technique is highly efficient and there is minimal interaction of the UDGx enzyme or complexes with other components used in downstream processes such as PCR or sequencing. Therefore, the step of adding UDGx can simply be added into existing enzymatic polynucleotide synthesis processes, and requires no further modifications to these methods.

Furthermore, the inventors have realised that the strong bond of the UDGx enzyme to uracil containing polynucleotides can be useful in other ways, such as a linker to attach polynucleotides to biological molecules or inert surfaces. Such uses of these enzymes have not before been envisaged.

Further features and embodiments of the above defined aspects will now be described under each of the following headed sections in the detailed description. The headed sections are not limiting, any feature in any of the sections may be combined with any aspect or embodiment herein in any workable combination.

### DETAILED DESCRIPTION OF THE INVENTION

### Polynucleotide

The present invention refers to removing or blocking uracil containing polynucleotides from compositions containing polynucleotides.

The term ‘polynucleotide’ as used herein refers to a polymer of nucleotides. Suitably to a polymer of A, T, U, G or C nucleotides, or optionally modified or synthetic nucleotides or nucleotide analogues comprising for example a modified bond, a modified purine or pyrimidine base, or a modified sugar. The terms “polynucleotide(s)”, “nucleic acid sequence(s)”, “nucleotide sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length.

The term ‘uracil containing’ with respect to a polynucleotide means that the polynucleotide contains at least one uracil nucleotide. In some cases, the polynucleotide may contain a plurality of uracil nucleotides. Suitably the ‘uracil containing polynucleotide’ contains or contained at least one uracil nucleotide before contact with the uracil DNA glycosylase enzyme described herein. Suitably such a term applies herein to the polynucleotide both before and after uracil removal by the uracil DNA glycosylase enzyme. Suitably one uracil DNA glycosylase enzyme may stably bind to one uracil nucleotide. Suitably therefore the uracil containing polynucleotide need only contain one uracil nucleotide to be bound by the uracil DNA glycosylase enzyme.

In one or more embodiments, the polynucleotide may be deoxyribonucleic acid (DNA) or ribonucleic acid (RNA), or in some cases may be a hybrid of DNA and RNA. In one or more embodiments, the polynucleotide may be an artificial polynucleotide or nucleic acid analogue selected from PNA, LNA, GNA, TNA, and HNA. In one or more embodiments, the polynucleotide may be a DNA or RNA molecule. In one or more embodiments, the polynucleotide may be single stranded (ss) or double stranded (ds). In one or more embodiments, the polynucleotide may be selected from ssRNA, ssDNA, dsRNA, and dsDNA. In one or more embodiments, the polynucleotide is single stranded. In one or more embodiments, the polynucleotide is DNA.

In one or more embodiments, the polynucleotide is single stranded DNA (ssDNA).

In one or more embodiments, the uracil containing polynucleotide may be uracil containing ssDNA. In one or more embodiments, the composition containing at least one polynucleotide may contain at least one ssDNA polynucleotide, preferentially a plurality of ssDNA polynucleotides. In one or more embodiments, the composition containing at least one polynucleotide may also contain other types of polynucleotides, it may sometimes contain a mixture of different types of polynucleotides. In one or more embodiments, the composition containing at least one polynucleotide may contain only ssDNA polynucleotides.

The present invention refers to at least one uracil containing polynucleotide, and compositions containing at least one polynucleotide.

The term ‘a’, or ‘at least one’ as used herein refers to one or more, more than one, or a plurality of the relevant feature. Therefore the at least one polynucleotide may be a plurality of polynucleotides. Suitably the compositions referred to herein contain a plurality of polynucleotides.

In one or more embodiments, the methods and uses of the invention comprise removing or blocking at least one uracil containing polynucleotide from a composition containing a plurality of polynucleotides. In one or more embodiments, the methods and uses of the invention comprise removing or blocking at least one uracil containing ssDNA polynucleotide from a composition containing a plurality of ssDNA polynucleotides.

In one or more embodiments, the methods and uses remove or block at least one uracil containing polynucleotide from the composition. In one or more embodiments, the methods and uses may remove or block a plurality of uracil containing polynucleotides from the composition. In one or more embodiments, the methods and uses may remove or block the majority of uracil containing polynucleotides from the composition. In one or more embodiments, the methods and uses may remove or block at least 50%, at least 55%, at least 60%, at least 65%, at least 70%, at least 75%, at least 80%, at least 85%, at least 90%, at least 95% at least 96%, at least 97%, at least 98%, at least 99% of uracil containing polynucleotides from the composition. In one or more embodiments, the methods and uses may remove or block substantially all of the uracil containing polynucleotides from the composition.

It will be understood that the composition containing at least one polynucleotide may or may not comprise uracil containing polynucleotides. In one or more embodiments, the methods and uses remove or block uracil containing polynucleotides from the composition, if they are present in the composition. In one or more embodiments, the uracil DNA glycosylase enzyme may be used for the purposes described herein and may fulfil said uses of the aspects of the invention, if it is contacted with a composition containing at least one polynucleotide, even if the composition does not contain any uracil containing polynucleotides.

The polynucleotides referred to herein may be of any length. Preferentially, the polynucleotides referred to herein may be up to 1000 nucleotides in length, more preferentially up to 200 nucleotides, more preferentially up to 100 bp, more preferentially between 5 to 100 nucleotides in length, more preferentially between 10 to 50 nucleotides in length, more preferentially between 10 to 35 nucleotides in length, more preferentially between 10 to 30 nucleotides in length, preferentially between 10 to 25 nucleotides in length, suitably between 10 to 20 nucleotides in length.

In one or more embodiments, therefore, the polynucleotides may be oligonucleotides.

### Uracil DNA Glycosylase Enzyme

The present invention makes use of particular uracil DNA glycosylase enzymes which form a stable bond with uracil containing polynucleotides which allows the uracil containing polynucleotides to be blocked or removed from compositions.

In one or more embodiments, uracil DNA glycosylase enzymes may be known as UNG or UDG as used herein.

In one or more embodiments, a ‘stable bond’ as used herein is a bond which persists for at least several hours, preferentially for at least a day, more preferentially for at least several days, more preferentially for at least a week, more preferentially for at least several weeks, more preferentially for at least a month, more preferentially for at least several months, more preferentially for at least a year, more preferentially for at least several years, without dissociating. more preferentially the stable bond persists indefinitely without dissociating.

In one or more embodiments, the stable bond may be any type of strong bond, such as a covalent bond, an ionic bond, or a hydrogen bond.

In one or more embodiments, the stable bond is a covalent bond. In one or more embodiments, the uracil DNA glycosylase enzyme is capable of forming a covalent bond with a uracil containing polynucleotide.

In one or more embodiments, the uracil DNA glycosylase enzyme is capable of binding to uracil, preferentially to a uracil nucleotide, more preferentially to a uracil nucleotide within the uracil containing polynucleotide. In one or more embodiments, each uracil DNA glycosylase enzyme is capable of forming a stable bond with a uracil nucleotide in a uracil containing polynucleotide.

In some or more embodiments, the uracil DNA glycosylase enzyme is capable of binding to an abasic site, preferentially to an abasic site after a uracil nucleotide is removed from the uracil containing polynucleotide.

In one or more embodiments, the uracil DNA glycosylase enzyme is capable of forming a covalent bond with an abasic site in the uracil containing polynucleotide. In one or more embodiments, each uracil DNA glycosylase enzyme is capable of forming a covalent bond with an abasic site after removal of a uracil nucleotide in a uracil containing polynucleotide.

In one or more embodiments, the uracil DNA glycosylase enzyme is a ‘UDGx’ enzyme. In one or more embodiments, UDGx enzymes excise uracil nucleotides from polynucleotides but do not dissociate from the polynucleotide and instead remain stably bound to uracil containing polynucleotide, preferentially to the remaining abasic site of the polynucleotide. In one or more embodiments, the UDG enzymes referred to herein and used in the methods of the invention are UDGx enzymes.

In one or more embodiments, the uracil DNA glycosylase enzyme may be a modified UDG enzyme, suitably which has been modified to remain stably bound to the uracil containing polynucleotide. Typically, UDG enzymes excise uracil nucleotides from polynucleotides and dissociate from the polynucleotide, they do not stably bind to the polynucleotide. In one or more embodiments, therefore the UDG enzyme may have one or more modifications which increases its binding affinity for a uracil containing polynucleotide. In one or more embodiments, the one or more modifications may be made in the UDG active site. In one or more embodiments, modifications may include those in or in proximity to the water activating loop of the UDG enzyme structure. Suitable modifications may include one or more amino acid substitutions in the water activating loop of the UDG enzyme or in proximity thereto.

A “substitution” means that an amino acid residue is replaced by another amino acid residue. In one or more embodiments, any amino acid may be used for the substitution. Suitably any proteinogenic amino acid may be used for the substitution. In one or more embodiments, the substitution is a conservative substitution as defined elsewhere herein.

In one or more embodiments, such modifications may include one or more amino acid substitutions at positions 109, 178 and/or 52 with reference to the M. smegmatis UDG amino acid sequence, or at corresponding positions thereto, as described in Ahn et al. Nature Chemical Biology, Volume 15, June 2019. Suitably the amino acid substitutions may insert a histidine at positions 109 and 178 i.e., 109H, 178H and/or insert a glutamic acid at position 52 i.e., 52E. In one or more embodiments, the UDG enzyme comprises an amino acid substitution at position 109 with reference to the M. smegmatis UDG amino acid sequence, or at a corresponding position thereto. In one or more embodiments, the UDG enzyme comprises amino acid substitution 109H with reference to the M. smegmatis UDG amino acid sequence, or at a corresponding position thereto.

In one or more embodiments, the uracil DNA glycosylase enzyme is a ‘UDGx’ enzyme.

In one or more embodiments, the uracil DNA glycosylase enzyme may be selected from any UDG enzyme derived from any organism.

In one or more embodiments, the uracil DNA glycosylase enzyme may be derived from a prokaryotic organism, such as a bacterium or an archaeon. Suitably the uracil DNA glycosylase enzyme may be derived from a bacterium. Suitably the uracil DNA glycosylase enzyme may be derived from a bacterium selected from the following genera: Streptomyces, Nocardia, Gordonia, Xanthomonas, Thiobacillus, Rhizobium, Bradyrhizobium, Mycobacterium, Gandjariella, Rhodococcus, and Amycolatopsis, for example. Suitably the uracil DNA glycosylase enzyme may be derived from a bacterium of one of the following species: Mycobacterium avium, Mycobacterium haemophilusobacterium chubense, Streptomyces coelicolor, Rhodococcus spp, Nocardia farcinica, Gordonia naminbiensis, Xanthomonas axonopodis, Thiobacillus denitrificans, Rhizobium leguminosarum, Bradyrhizobium japonicum, Mycolicibacterium smegmamtis, Mycolicibacterium thermoresistable, Mycobacterium colombiense, Gandjariella thermophilia, Rhodococcus ruber, Rhodococcus rhodochrous, and Amycolatopsis viridis, for example.

In one or more embodiments, the uracil DNA glycosylase enzyme is derived from a bacterium selected from the following species: Mycolicibacterium thermoresistable, Mycobacterium colombiense, and Rhodococcus rhodochrous.

In one or more embodiments, the uracil DNA glycosylase enzyme is a ‘UDGx’ enzyme derived from a bacterium selected from Mycolicibacterium thermoresistable, Mycobacterium colombiense, and Rhodococcus rhodochrous.

In one or more embodiments, the uracil DNA glycosylase enzyme comprises a sequence selected from any of SEQ ID NO: 1-12, or a sequence having at least 70% identity thereto.

In one or more embodiments, any sequence referred to herein as having at least 70% identity to a reference sequence may have at least 75%, at least 80%, at least 85%, at least 90%, at least 95%, at least 96%, at least 97%, at least 98%, at least 99% identity to the reference sequence.

In one or more embodiments, the uracil DNA glycosylase enzyme may consist of a sequence selected from any of SEQ ID NO: 1-12.

“Identity” or “percent identity” refers to the degree of sequence variation between two given nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of (Smith and Waterman, 1981), by the homology alignment algorithm of (Needleman and Wunsch, 1970), by the search for similarity method of (Pearson and Lipman, 1988), by computerized implementations of these algorithms (GAP, BESTFIT, FASTA, and TFASTA in the Wisconsin Genetics Software Package, Genetics Computer Group, 575 Science Dr., Madison, WI), or by visual inspection. One example of an algorithm that is suitable for determining percent sequence identity and sequence similarity is the BLAST algorithm, which is described in (Altschul et al., 1990). Software for performing BLAST analyses is publicly available through the National Center for Biotechnology Information (on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., 1990) These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix ((Henikoff and Henikoff, 1992). In addition to calculating percent sequence identity, the BLAST algorithm also performs a statistical analysis of the similarity between two sequences (Karlin and Altschul, 1990). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

In one or more embodiments, the uracil DNA glycosylase enzyme may be modified, and may be regarded as a variant or mutant. In one or more embodiments, the uracil DNA glycosylase enzyme may comprise one or more modifications thereto to improve stability. In one or more embodiments, the uracil DNA glycosylase enzyme may comprise one or more amino acid modifications.

In one or more embodiments, such modifications may be amino acid modifications selected from substitutions, insertions or deletions. In one or more embodiments, the modification is a substitution of the amino acid at the recited position with a different amino acid.

A “substitution” means that an amino acid residue is replaced by another amino acid residue. In one or more embodiments, any amino acid may be used for the substitution. In one or more embodiments, any proteinogenic amino acid may be used for the substitution. In one or more embodiments, the substitution is a conservative substitution.

In one or more embodiments, such modified uracil DNA glycosylase enzymes may be obtained by various techniques well known in the art. In particular, examples of techniques for altering the DNA sequence encoding the wild-type protein, include, but are not limited to, site-directed mutagenesis, random mutagenesis and synthetic oligonucleotide construction. The term “wild-type”, as used herein refers to the non-mutated version of a nucleic acid or protein as it appears naturally. In one or more embodiments, the wild type uracil DNA glycosylase enzymes from which the modified forms of the invention are derived comprise the sequences in SEQ ID NO: 2, 7 or 9 defined herein.

In one or more embodiments, modified uracil DNA glycosylase enzymes of the invention may be produced by mutating wild type uracil DNA glycosylase-coding polynucleotides, then expressing the modified polynucleotides using conventional molecular biology techniques. For example, a desired gene or DNA fragment encoding a uracil DNA glycosylase polypeptide of desired sequence may be assembled from synthetic fragments using conventional molecular biology techniques, e.g. using protocols described by Stemmer et al, Gene, 164:49-53 (1995); Kodumal et al, Proc. Natl. Acad. Sci., 101:15573-15578 (2004); or the like, or such gene or DNA fragment may be directly cloned from cells of a selected species using conventional protocols.

An isolated gene encoding a desired modified uracil DNA glycosylase enzyme may be inserted into an expression vector to give an expression vector which then may be used to make and express the modified uracil DNA glycosylase protein using conventional methods. Such vectors may be transformed into producer strains such as E. coli.

In one or more embodiments, the transformed strains are then cultured using conventional techniques to form a population of transformed cells from which the modified uracil DNA glycosylase enzyme is extracted. For example, the cultured cells may be exposed to lysis buffer composed of 50 mM tris-HCL (Sigma) pH 7.5, 150 mM NaCl (Sigma), 0.5 mM mercaptoethanol (Sigma), 5% glycerol (Sigma), 20 mM imidazole (Sigma) and 1 tab for 100 mL of protease cocktail inhibitor (Thermofisher). The cells may then be lysed by any known means, such as through several cycles of French press, until full color homogeneity is obtained, at a typical pressure of 14,000 psi. The obtained cell lysate is then centrifuged for Ih to Ih 30 at 10,000 rpm. Centrifugate is then passed through a 0.2 μm filter to remove any debris before column purification.

Modified uracil DNA glycosylase enzymes may be purified from the centrifugate in a one-step affinity procedure. For example, Ni-NTA affinity column (GE Healthcare) may be used to bind the uracil DNA glycosylase enzymes. Initially the column is washed and equilibrated with 15 column volumes of 50 mM tris-HCL (Sigma) pH 7.5, 150 mM NaCl (Sigma) and 20 mM imidazole (Sigma). uracil DNA glycosylase enzymes are bound to the column after equilibration; then, a washing buffer, for example, composed of 50 mM tris-HCL (Sigma) pH 7.5, 500 mM NaCl (Sigma) and 20 mM imidazole (Sigma), may be applied to the column for 15 column volumes. After such washing, the enzymes are eluted with 50 mM tris-HCL (Sigma) pH 7.5, 500 mM NaCl (Sigma) and 0.5M imidazole (Sigma). Fractions corresponding to the highest concentration of modified uracil DNA glycosylase enzymes of interest are collected and pooled in a single sample. The pooled fractions are dialyzed against the dialysis buffer (20 mM Tris-HCl, pH 6.8, 200 mM Na CI, 50 mM MgOAc, 100 mM [NH4]2S04). The dialysate is subsequently concentrated with the help of concentration filters (Amicon Ultra-30, Merk Millipore). Concentrated enzyme is distributed in small aliquots, 50% glycerol final is added, and those aliquots are then frozen at −20° C. and stored for long term. 5 pL of various fraction of the purified enzymes are analysed in SDS-PAGE gels.

By ‘conservative’ it is meant that an amino acid with similar characteristics may be used for the substitution. Conservative amino acid substitutions” refer to the interchangeability of residues having similar side chains, and thus typically involves substitution of an amino acid in a polypeptide with amino acids within the same or similar defined class of amino acids. By way of example, an amino acid with an aliphatic side chain may be substituted with another aliphatic amino acid, e.g., alanine, valine, leucine, and isoleucine; an amino acid with hydroxyl side chain may be substituted with another amino acid with a hydroxyl side chain, e.g., serine and threonine; an amino acids having aromatic side chains may be substituted with another amino acid having an aromatic side chain, e.g., phenylalanine, tyrosine, tryptophan, and histidine; an amino acid with a basic side chain may be substituted with another amino acid with a basic side chain, e.g., lysine and arginine; an amino acid with an acidic side chain may be substituted with another amino acid with an acidic side chain, e.g., aspartic acid or glutamic acid; and a hydrophobic or hydrophilic amino acid may be substituted with another hydrophobic or hydrophilic amino acid, respectively.

In one or more embodiments, the uracil DNA glycosylase enzyme comprises one or more modifications that are ancestral modifications, preferentially said modifications are ancestral substitution mutations. In one or more embodiments, the uracil DNA glycosylase enzyme comprises one or more ancestral amino acid substitution mutations. In one or more embodiments, ancestral mutations are defined as mutations which return the amino acid residues back to those which were present at specific positions in a predicted ancestral amino acid sequence for said protein at the cluster nodes of a phylogenetic tree, wherein the predicted ancestral amino acid sequence of said protein is derived from the alignment of many existing protein sequences belonging to the same protein family. In one or more embodiments, ancestral mutations may be identified by ancestral sequence reconstruction, suitable tools to identify ancestral mutations are known in the art, for example: BEAST, Network, MEGA6, and FireProt ASR. In one or more embodiments, ancestral substitution mutations are defined as substitutions which return the amino acid residues back to those which were present at specific positions by substitution in a predicted ancestral amino acid sequence for said protein at the cluster nodes of a phylogenetic tree, wherein the predicted ancestral amino acid sequence of said protein is derived from the alignment of many existing protein sequences belonging to the same protein family.

In one or more embodiments, therefore, the uracil DNA glycosylase enzyme comprises an amino acid sequence selected from any of SEQ ID NO: 2, 7 and 9, or a sequence having at least 70% identity thereto, having or comprising one or more modifications. In one or more embodiments, one or more mutations. In one or more embodiments, one or more ancestral mutations. In one or more embodiments, one or more substitution mutations. In one or more embodiments, one or more ancestral substitution mutations.

In one or more embodiments, therefore, the uracil DNA glycosylase enzyme comprises an amino acid sequence selected from any of SEQ ID NO: 2, 7 and 9, or a sequence having at least 75% identity thereto, having or comprising one or more modifications. In one or more embodiments, one or more mutations. In one or more embodiments, one or more ancestral mutations. In one or more embodiments, one or more substitution mutations. In one or more embodiments, one or more ancestral substitution mutations.

In one or more embodiments, therefore, the uracil DNA glycosylase enzyme comprises an amino acid sequence selected from any of SEQ ID NO: 2, 7 and 9, or a sequence having at least 80% identity thereto, having or comprising one or more modifications. In one or more embodiments, one or more mutations. In one or more embodiments, one or more ancestral mutations. In one or more embodiments, one or more substitution mutations. In one or more embodiments, one or more ancestral substitution mutations.

In one or more embodiments, therefore, the uracil DNA glycosylase enzyme comprises an amino acid sequence selected from any of SEQ ID NO: 2, 7 and 9, or a sequence having at least 85% identity thereto, having or comprising one or more modifications. In one or more embodiments, one or more mutations. In one or more embodiments, one or more ancestral mutations. In one or more embodiments, one or more substitution mutations. In one or more embodiments, one or more ancestral substitution mutations.

In one or more embodiments, therefore, the uracil DNA glycosylase enzyme comprises an amino acid sequence selected from any of SEQ ID NO: 9, or a sequence having at least 90% identity thereto, having or comprising one or more modifications. In one or more embodiments, one or more mutations. In one or more embodiments, one or more ancestral mutations. In one or more embodiments, one or more substitution mutations. In one or more embodiments, one or more ancestral substitution mutations.

The amino acids are herein represented by their one-letter or three-letters code according to the standard international nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (He); K: lysine (Lys); L: leucine (Leu); M: methionine (Met); N: asparagine (Asn); P: proline (Pro); Q: glutamine (Gin); R: arginine (Arg); S: serine (Ser); T: threonine (Thr); V: valine (Vai); W: tryptophan (Trp) and Y: tyrosine (Tyr).

In one or more embodiments, the uracil DNA glycosylase enzyme comprises an amino acid sequence according to SEQ ID NO:2 or a functionally equivalent sequence or a fragment thereof, or preferably a sequence having at least 70%, 75%, 80%, 85% identity thereto, wherein the amino acid sequence comprises mutations at one or more of the following positions: A12, V23, A25, V33, A38, T46, V50, M51, R59, T62, Q76, D80, A81, E88, T99, R104, L106, S114, D115, L137, K144, L153, G166, G171, L172, G173, L177, G193, and E207, or corresponding positions thereto.

In one or more embodiments, the uracil DNA glycosylase enzyme comprises an amino acid sequence according to SEQ ID NO:2, or a functionally equivalent sequence or a fragment thereof, or preferably a sequence having at least 70%, 75%, 80%, 85% identity thereto, wherein the amino acid sequence comprises mutations at each of the following positions: A12, V23, A25, V33, A38, T46, V50, M51, R59, T62, Q76, D80, A81, E88, T99, R104, L106, S114, D115, L137, K144, L153, G166, G171, L172, G173, L177, G193, and E207, or corresponding positions thereto.

In one or more embodiments, the uracil DNA glycosylase enzyme comprises an amino acid sequence according to SEQ ID NO:2, or a functionally equivalent sequence or a fragment thereof, or preferably a sequence having at least 70%, 75%, 80%, 85% identity thereto wherein the amino acid sequence comprises one or more of the following mutations: A12E, V23A, A25G, V33R, A38T, T46S, V50M, M51L, R59Q, T62R, Q76D, D80E, A81E, E88Q, T99K, R104K, L106R, S114T, D115E, L137C, K144Q, L153V, G166E, G171T, L172V, G173D, L177R, G193E, and E207R, or one or more of said mutations at corresponding positions thereto.

In one or more embodiments, the uracil DNA glycosylase enzyme comprises an amino acid sequence according to SEQ ID NO:2, or a functionally equivalent sequence or a fragment thereof, or preferably a sequence having at least 70%, 75%, 80%, 85% identity thereto wherein the amino acid sequence comprises each of the following mutations: A12E, V23A, A25G, V33R, A38T, T46S, V50M, M51L, R59Q, T62R, Q76D, D80E, A81E, E88Q, T99K, R104K, L106R, S114T, D115E, L137C, K144Q, L153V, G166E, G171T, L172V, G173D, L177R, G193E, and E207R, or each of said mutations at corresponding positions thereto.

In one or more embodiments, the uracil DNA glycosylase enzyme comprises an amino acid sequence according to SEQ ID NO:2, or a functionally equivalent sequence or a fragment thereof, or preferably a sequence having at least 85% identity thereto wherein the amino acid sequence comprises each of the following mutations: A12E, V23A, A25G, V33R, A38T, T46S, V50M, M51L, R59Q, T62R, Q76D, D80E, A81E, E88Q, T99K, R104K, L106R, S114T, D115E, L137C, K144Q, L153V, G166E, G171T, L172V, G173D, L177R, G193E, and E207R, or each of said mutations at corresponding positions thereto.

In one or more embodiments, the uracil DNA glycosylase enzyme comprises an amino acid sequence according to SEQ ID NO:7, or a functionally equivalent sequence or a fragment thereof, or preferably a sequence having at least 70%, 75%, 80%, 85% identity thereto wherein the amino acid sequence comprises mutations at one or more of the following positions: A11, G19, M31, M92, E95, Q139, A155, T156, T161, S171, T172, H176, T178, L183, V203, E205, A206, A216, R220, and G222 or corresponding positions thereto.

In one or more embodiments, the uracil DNA glycosylase enzyme comprises an amino acid sequence according to SEQ ID NO:7, or a functionally equivalent sequence or a fragment thereof, or preferably a sequence having at least 70%, 75%, 80%, 85% identity thereto wherein the amino acid sequence comprises mutations at each of the following positions: A11, G19, M31, M92, E95, Q139, A155, T156, T161, S171, T172, H176, T178, L183, V203, E205, A206, A216, R220, and G222, or corresponding positions thereto.

In one or more embodiments, the uracil DNA glycosylase enzyme comprises an amino acid sequence according to SEQ ID NO:7, or a functionally equivalent sequence or a fragment thereof, or preferably a sequence having at least 70%, 75%, 80%, 85% identity thereto wherein the amino acid sequence comprises one or more of the following mutations: A11D, G19D, M31R, M92I, E95V, Q139E, A155S, T156D, T161A, S171A, T172S, H176D, T178A, L183V, V203A, E205Q, A206S, A216G, R220G, and G222A or one or more of said mutations at corresponding positions thereto.

In one or more embodiments, the uracil DNA glycosylase enzyme comprises an amino acid sequence according to SEQ ID NO:7, or a functionally equivalent sequence or a fragment thereof, or preferably a sequence having at least 70%, 75%, 80%, 85% identity thereto wherein the amino acid sequence comprises each of the following mutations: A11D, G19D, M31R, M92I, E95V, Q139E, A155S, T156D, T161A, S171A, T172S, H176D, T178A, L183V, V203A, E205Q, A206S, A216G, R220G, and G222A or each of said mutations at corresponding positions thereto.

In one or more embodiments, the uracil DNA glycosylase enzyme comprises an amino acid sequence according to SEQ ID NO:7, or a functionally equivalent sequence or a fragment thereof, or preferably a sequence having at least 85% identity thereto wherein the amino acid sequence comprises each of the following mutations: A11D, G19D, M31R, M92I, E95V, Q139E, A155S, T156D, T161A, S171A, T172S, H176D, T178A, L183V, V203A, E205Q, A206S, A216G, R220G, and G222A, or each of said mutations at corresponding positions thereto.

In one or more embodiments, the uracil DNA glycosylase enzyme comprises an amino acid sequence according to SEQ ID NO:9, or a functionally equivalent sequence or a fragment thereof, or preferably a sequence having at least 70%, 75%, 80%, 85%, 90% identity thereto wherein the amino acid sequence comprises mutations at one or more of the following positions: G10, T17, S27, N37, E39, R40, L52, V53, G90, E91, E104, A107, A108, G110, A118, G120, I166, P169, D170, I173, P174, A178, I183, I188, Q195, E197, L200, and G202, or corresponding positions thereto.

In one or more embodiments, the uracil DNA glycosylase enzyme comprises an amino acid sequence according to SEQ ID NO:9, or a functionally equivalent sequence or a fragment thereof, or preferably a sequence having at least 70%, 75%, 80%, 85%, 90% identity thereto wherein the amino acid sequence comprises mutations at each of the following positions: G10, T17, S27, N37, E39, R40, L52, V53, G90, E91, E104, A107, A108, G110, A118, G120, I166, P169, D170, I173, P174, A178, I183, I188, Q195, E197, L200, and G202, or corresponding positions thereto.

In one or more embodiments, the uracil DNA glycosylase enzyme comprises an amino acid sequence according to SEQ ID NO:9, or a functionally equivalent sequence or a fragment thereof, or preferably a sequence having at least 70%, 75%, 80%, 85%, 90% identity thereto wherein the amino acid sequence comprises one or more of the following mutations: G10A, T17R, S27R, N37D, E39T, R40Q, L52M, V53M, G90E, E91R, E104T, A107E, A108G, G110K, A118S, G120T, I166V, P169L, D170P, I173V, P174E, A178R, I183V, I188V, Q195D, E197D, L200F, and G202A, or one or more of said mutations at corresponding positions thereto.

In one or more embodiments, the uracil DNA glycosylase enzyme comprises an amino acid sequence according to SEQ ID NO:9, or a functionally equivalent sequence or a fragment thereof, or preferably a sequence having at least 70%, 75%, 80%, 85%, 90% identity thereto wherein the amino acid sequence comprises each of the following mutations: G10A, T17R, S27R, N37D, E39T, R40Q, L52M, V53M, G90E, E91R, E104T, A107E, A108G, G110K, A118S, G120T, I166V, P169L, D170P, I173V, P174E, A178R, I183V, I188V, Q195D, E197D, L200F, and G202A, or each of said mutations at corresponding positions thereto.

In one or more embodiments, the uracil DNA glycosylase enzyme comprises an amino acid sequence according to SEQ ID NO:9, or a functionally equivalent sequence or a fragment thereof, or preferably a sequence having at least 90% identity thereto wherein the amino acid sequence comprises each of the following mutations: G10A, T17R, S27R, N37D, E39T, R40Q, L52M, V53M, G90E, E91R, E104T, A107E, A108G, G110K, A118S, G120T, I166V, P169L, D170P, I173V, P174E, A178R, I183V, I188V, Q195D, E197D, L200F, and G202A, or each of said mutations at corresponding positions thereto.

‘Functional equivalent sequence’ refers to a sequence homologous to the disclosed sequence and having an identical functional role.

‘Corresponding position thereto’ as used herein means the same amino acid position in a different reference sequence, suitably in a different uracil DNA glycosylase enzyme sequence. Therefore, whilst the statements herein refer to certain SEQ ID NOs, the invention is not restricted to the uracil DNA glycosylase enzyme of said SEQ ID NOs, each modification may be located at a position corresponding to an amino acid position denoted above in another uracil DNA glycosylase enzyme sequence. Therefore, the invention equally refers to other uracil DNA glycosylase enzymes having different amino acid sequences with the same modifications. It is possible to compare uracil DNA glycosylase polypeptides by sequence comparison and locate conserved regions that correspond to the amino acid positions listed above. Sequence comparison to find corresponding positions may be carried out by aligning the amino acid sequences of two or more proteins, using an alignment program such as BLAST®. Methods for the alignment of sequences for comparison are well known in the art, such methods include GAP, BESTFIT, BLAST, FASTA and TFASTA. GAP uses the algorithm of Needleman and Wunsch ((1970) J Mol Biol 48:443-453) to find the global (i.e. spanning the complete sequences) alignment of two sequences that maximizes the number of matches and minimizes the number of gaps. The BLAST algorithm (Altschul et al. (1990) J Mol Biol 215:403-10) calculates percent sequence identity and performs a statistical analysis of the similarity between the two sequences. The software for performing BLAST analysis is publicly available through the National Centre for Biotechnology Information (NCBI). Homologues may readily be identified using, for example, the ClustalW multiple sequence alignment algorithm (version 1.83), with the default pairwise alignment parameters, and a scoring method in percentage. Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences.). Minor manual editing may be performed to optimise alignment between conserved motifs, as would be apparent to a person skilled in the art. Furthermore, instead of using full-length sequences for the identification of homologues, specific domains may also be used. The sequence identity values may be determined over the entire nucleic acid or amino acid sequence or over selected domains or conserved motif(s), using the programs mentioned above using the default parameters. For local alignments, the Smith-Waterman algorithm is particularly useful (Smith T F, Waterman M S (1981) J. Mol. Biol 147 (1); 195-7). In the present case, a corresponding position in a different uracil DNA glycosylase enzyme sequence may be found by aligning the amino acid sequence of said other uracil DNA glycosylase enzyme with SEQ ID NOs 2, 7 or 9, and locating the same amino acid position as those listed.

In one or more embodiments, the uracil DNA glycosylase enzyme comprises an amino acid sequence selected from any of SEQ ID NO: 6, 8 and 10, or a sequence having at least 70%, 75%, 80%, 85%, 90%, 95%, 96%, 97%, 98%, 99% identity thereto. Suitably wherein the mutations present in these sequences are retained. Suitably wherein the mutations present in these sequences compared to the wild type sequences according to SEQ ID NOs: 2, 7 and 9 respectively are retained.

In one or more embodiments, the uracil DNA glycosylase enzyme comprises an amino acid sequence according to SEQ ID NO: 6, or a sequence having at least 70%, 75%, 80%, 85%, 90%, 95%, 96%, 97%, 98%, 99% identity thereto which retains/comprises each of the following mutations: A12E, V23A, A25G, V33R, A38T, T46S, V50M, M51L, R59Q, T62R, Q76D, D80E, A81E, E88Q, T99K, R104K, L106R, S114T, D115E, L137C, K144Q, L153V, G166E, G171T, L172V, G173D, L177R, G193E, and E207R.

In one or more embodiments, the uracil DNA glycosylase enzyme comprises an amino acid sequence according to SEQ ID NO: 8, or a sequence having at least 70%, 75%, 80%, 85%, 90%, 95%, 96%, 97%, 98%, 99% identity thereto which retains/comprises each of the following mutations: A11D, G19D, M31R, M92I, E95V, Q139E, A155S, T156D, T161A, S171A, T172S, H176D, T178A, L183V, V203A, E205Q, A206S, A216G, R220G, and G222A.

In one or more embodiments, the uracil DNA glycosylase enzyme comprises an amino acid sequence according to SEQ ID NO: 10, or a sequence having at least 70%, 75%, 80%, 85%, 90%, 95%, 96%, 97%, 98%, 99% identity thereto which retains/comprises each of the following mutations: G10A, T17R, S27R, N37D, E39T, R40Q, L52M, V53M, G90E, E91R, E104T, A107E, A108G, G110K, A118S, G120T, I166V, P169L, D170P, I173V, P174E, A178R, I183V, I188V, Q195D, E197D, L200F, and G202A.

In one or more embodiments, the uracil DNA glycosylase enzyme consists of a sequence selected from any of SEQ ID NO: 6, 8 and 10.

### Binding Molecule and Capture Partner

In one or more embodiments of the present invention, the uracil DNA glycosylase enzyme may comprise a binding molecule to enable the enzyme and uracil containing polynucleotide complexes to be removed from compositions containing polynucleotides.

In one or more embodiments, the binding molecule is attached to the uracil DNA glycosylase enzyme, suitably by any means, for example via a linker. In one or more embodiments, the binding molecule is fused to the uracil DNA glycosylase enzyme. In one or more embodiments, therefore the uracil DNA glycosylase enzyme is expressed as a fusion protein with the binding molecule.

In one or more embodiments, the binding molecule may be attached to either the N or the C terminus of the uracil DNA glycosylase enzyme. In one or more embodiments, the binding molecule is attached to the C-terminus of the uracil DNA glycosylase enzyme. Alternatively, in some cases, there may be two binding molecules, each attached to the N and the C terminus of the uracil DNA glycosylase enzyme. In such cases, the two binding molecules are suitably different.

In one or more embodiments, the binding molecule is any molecule which enables the isolation of enzyme-uracil containing polynucleotide complexes.

In one or more embodiments, the binding molecule may be selected from a silica binding tag, a His-tag, a streptactin or Strep-tag, a biotin tag, a streptavidin tag, a cellulose binding domain, MBP, GST and the like. In one or more embodiments, the binding molecule may also be an antigen, suitably an inert antigen. In one or more embodiments, therefore the binding molecule may be a tag, suitably a tag selected from a silica binding tag, a streptactin tag/Strep tag, a His-tag, and a streptavidin tag.

In one or more embodiments, the binding molecule may have a capture partner. In one or more embodiments, the capture partner is capable of binding to the binding molecule and capturing it.

In one or more embodiments, the capture partner is chosen to match the chosen binding molecule. In one or more embodiments, the capture partner is selected from: silica, metal chelation resin such as Ni-NTA or TALON, streptavidin, Streptactin-Sepharose (IBA), avidin, cellulose, maltose, and glutathione, respectively. In one or more embodiments, the capture partner may also be an antibody or antigen binding fragment thereof.

In one or more embodiments, the binding molecule is a His Tag, and the capture molecule is Ni-NTA.

In one or more embodiments, the binding molecule is a cellulose binding domain, and the capture molecule is cellulose. In one or more embodiments, the cellulose binding domain may be derived from Clostridium thermocellum.

In one or more embodiments, the binding molecule is a Streptactin/strep tag, and the capture molecule is Streptactin-Sepharose (IBA).

In one or more embodiments, the streptactin tag may comprise the following sequence: AWSHPQFEKGGGSGGGSGGSSAWSHPQFEK (SEQ ID NO:34)

In one or more embodiments, the binding molecule is a silica binding tag, and the capture molecule is silica, or in some cases where the silica binding tag contains a plurality of histidine residues, the capture molecule may also be Ni-NTA. In one or more embodiments, the silica capture molecule may be silica, or may be a silica derivative or compound such as SiO₂, Ge-on-Si, Quartz, silicic acid, or siliceous spicules.

Suitable silica binding tags are well known in the art, and may be selected from any of the following sequences: APPGHHHWHIHH (SEQ ID NO:35), MSASSYASFSWS (SEQ ID NO: 36), KPSHHHHHTGAN (SEQ ID NO:37), MSPHPHPRHHHT (SEQ ID NO:38), MSPHHMHHSHGH (SEQ ID NO:39), LPHHHHLHTKLP (SEQ ID NO:40), APHHHHPHHLSR (SEQ ID NO:41), RGRRRRLSCRLL (SEQ ID NO:42), HPPMNASHPHMH (SEQ ID NO:43), HTKHSHTSPPPL (SEQ ID NO:44), CHKKPSKSC (SEQ ID NO:45), CTSPHTRAC (SEQ ID NO: 46), CSYHRMATC (SEQ ID NO:47), RLNPPSQMDPPF (SEQ ID NO:48), QTWPPPLWFSTS (SEQ ID NO:49), YITPYAHLRGGN (SEQ ID NO:50), KSLSRHDHIHHH (SEQ ID NO:51), LDHSLHS (SEQ ID NO:52), MHRSDLMSAAVR (SEQ ID NO:53), KLPGWSG (SEQ ID NO:54), AFILPTG (SEQ ID NO:55), LSNNNLR (SEQ ID NO:56), AAPSHEHRHSRQ (SEQ ID NO:57), ALAHNPKTTHHR (SEQ ID NO:58), ERPLHIHYHKGQ (SEQ ID NO:59), and TTHSKHHFPSSA (SEQ ID NO:60).

In one or more embodiments, the uracil DNA glycosylase enzyme comprises a Streptactin/Strep tag, preferentially at its C-terminus.

In one or more embodiments, the uracil DNA glycosylase enzyme may further comprise a purification tag. In one or more embodiments, the purification tag may be attached to either the N or the C terminus of the uracil DNA glycosylase enzyme. In one or more embodiments, the purification tag is attached to the N-terminus of the uracil DNA glycosylase enzyme.

In one or more embodiments, the purification tag enables purification of the uracil DNA glycosylase enzyme during production thereof, preferentially during recombinant production thereof from a bacterial host. In one or more embodiments, the purification tag may be any tag such as those listed above, preferentially it is different to the binding molecule.

In one or more embodiments, the uracil DNA glycosylase enzyme comprises a His-tag, suitably at its N-terminus.

In one or more embodiments, therefore, the uracil DNA glycosylase enzyme comprises a His-tag at its N-terminus and a Streptactin/Strep tag at its C-terminus.

In one or more embodiments, the binding molecule may also act as a purification tag, therefore the uracil DNA glycosylase enzyme may only comprise one binding molecule at the N or C terminus thereof, which also acts as a purification tag.

In one or more embodiments, the capture partner may be immobilised, preferentially immobilised on a solid support. In one or more embodiments, the solid support may be a surface, a column or a bead for example. In some cases, the column may comprise one more beads upon which the capture partner may be immobilised.

In one or more embodiments, in methods or uses of the invention relating to removing at least one uracil containing polynucleotide from a composition containing at least one polynucleotide, the uracil DNA glycosylase enzyme is attached to or comprises a binding molecule. In one or more embodiments, such methods have a step of separating the one or more enzyme-U-polynucleotide complexes from the composition. In one or more embodiments, the step of separating may comprise removing the one or more enzyme-U-polynucleotide complexes from the composition, or removing the composition from the one or more enzyme-U-polynucleotide complexes.

By ‘enzyme-U-polynucleotide complex’ it is meant the complex of a uracil DNA glycosylase enzyme bound to a uracil containing polynucleotide. By ‘binding molecule-enzyme-U-polynucleotide complex’ it is meant the complex of a uracil DNA glycosylase enzyme comprising a binding molecule bound to a uracil containing polynucleotide.

In one or more embodiments, such a separating step may comprise contacting the composition with a capture partner, preferentially for capturing the binding molecule.

In one embodiment therefore, the method of removing at least one uracil containing polynucleotide from a composition containing at least one polynucleotide comprises the steps of:
(a) Providing a composition containing at least one polynucleotide;
(b) Contacting the composition with at least one uracil DNA glycosylase enzyme, wherein each enzyme is capable of forming a stable bond with a uracil containing polynucleotide and is further attached to a binding molecule, to form one or more binding molecule-enzyme-U-polynucleotide complexes;
(c) Contacting the composition with a capture partner which is capable of capturing the binding molecule;
(d) Separating the one or more captured binding molecule-enzyme-U-polynucleotide complexes from the composition

In one or more embodiments, contacting the composition with a capture partner may comprise contacting the composition with a solid support comprising the capture partner. In one or more embodiments, the capture partner is on an exposed surface of the solid support. In one or more embodiments, contacting the composition with a capture partner may comprise contacting the composition with a column and/or one or more beads comprising the capture partner, preferentially on an exposed surface thereof.

In one or more embodiments, the capture partner may be added sequentially to the addition of the uracil DNA glycosylase enzyme, preferentially before or after the addition of uracil DNA glycosylase enzyme. In one or more embodiments, the capture partner may be added after the addition of the uracil DNA glycosylase enzyme, as indicated above at step (c). In one or more embodiments, the capture partner may be added simultaneously with the uracil DNA glycosylase enzyme, preferentially both may be added in step (b). In one or more embodiments, step (b) may comprise contacting the composition with at least one uracil DNA glycosylase enzyme attached to a binding molecule and a capture partner.

In one or more embodiments, the uracil DNA glycosylase enzyme may be pre-bound to the capture partner. In one or more embodiments, the uracil DNA glycosylase enzyme is attached to a binding molecule which is attached to a capture partner. In one or more embodiments the methods comprises only step (a) of contacting the composition with at least one pre-bound uracil DNA glycosylase enzyme, wherein the prebound uracil DNA glycosylase enzyme is capable of forming a stable bond with a uracil containing polynucleotide and is attached to a binding molecule and a capture partner, to form one or more captured binding molecule-enzyme-U-polynucleotide complexes. In one or more embodiments the uracil DNA glycosylase enzyme is immobilised, suitably on a support as mentioned above. In one or more embodiments, the uracil DNA glycosylase enzyme may be immobilised on the support via a binding and capture partner, or by other means such as chemical attachment. Suitably therefore the uracil DNA glycosylase enzyme binds to a uracil containing DNA to form an enzyme-U-polynucleotide complex which is attached to the support, and can suitably be removed.

In one or more embodiments, the method of removing at least one uracil containing polynucleotide from a composition containing at least one polynucleotide comprises the steps of:
(a) Providing a composition containing at least one polynucleotide;
(b) Contacting the composition with a support comprising at least one uracil DNA glycosylase enzyme immobilised thereon, wherein each enzyme is capable of forming a stable bond with a uracil containing polynucleotide, to form one or more enzyme-U-polynucleotide complexes attached to the support;
(c) Separating the support comprising the one or more enzyme-U-polynucleotide complexes from the composition

In one or more embodiments, the support comprising the uracil DNA glycosylase enzyme immobilised thereon may be a bead or a column, for example. In one or more embodiments, the uracil DNA glycosylase enzyme maybe immobilised on the support by any means, preferentially by the above described binding molecules and capture partners.

In one or more embodiments where the composition comprising the enzyme is contacted with a capture partner, the composition may be contacted with a column comprising the capture partner. In one or more embodiments contacting with a column may comprise passing the composition through or over the column comprising the capture partner. In one or more embodiments in some cases, the column may comprise one or more beads therein comprising the capture partner. In one or more embodiments the capture partner captures the binding molecule and hence the binding molecule-enzyme-U-polynucleotide complexes. In one or more embodiments a purified composition is obtained from the column, preferentially the purified composition obtained from the column comprises fewer enzyme-U-polynucleotide complexes than the composition that entered the column, more preferentially the purified composition that is obtained from the column comprises substantially no enzyme-U-polynucleotide complexes. In one or more embodiments, such a method may further comprise one or more additional washing steps to wash the column. Optionally the step of passing the composition through the column may be repeated one or more times.

In one or more embodiments, the method of removing at least one uracil containing polynucleotide from a composition containing at least one polynucleotide comprises the steps of:
(a) Providing a composition containing at least one polynucleotide;
(b) Contacting the composition with at least one uracil DNA glycosylase enzyme wherein the enzyme is capable of forming a stable bond with a uracil containing polynucleotide and is further attached to a binding molecule, to form one or more binding molecule-enzyme-U-polynucleotide complexes;
(c) Passing the composition through or over a column comprising a capture partner which is capable of capturing the binding molecule;
(d) Obtaining a purified composition from the column

In one or more embodiments, contacting with one or more beads may comprise adding one or more beads comprising the capture partner to the composition. Suitably the capture partner captures the binding molecule and hence the binding molecule-enzyme-U-polynucleotide complexes. In one or more embodiments, the beads may then be separated from the composition, suitably by filtration or otherwise. In one or more embodiments, the remaining composition is a purified composition. In one or more embodiments, the purified composition comprises fewer enzyme-U-polynucleotide complexes than the composition that entered the column, preferentially the purified composition comprises substantially no enzyme-U-polynucleotide complexes. In one or more embodiments, such a method may further comprise one or more additional washing steps to wash the beads. Optionally the steps of adding one or more beads to the composition and separating the or each bead may be repeated one or more times.

In one or more embodiments, the method of removing at least one uracil containing polynucleotide from a composition containing at least one polynucleotide comprises the steps of:
(a) Providing a composition containing at least one polynucleotide;
(b) Contacting the composition with at least one uracil DNA glycosylase enzyme which is capable of forming a stable bond with a uracil containing polynucleotide and is further attached to a binding molecule, to form one or more binding molecule-enzyme-U-polynucleotide complexes;
(c) Adding one or more beads to the composition, the or each bead comprising a capture partner which is capable of capturing the binding molecule;
(d) Separating the or each bead from the composition to obtain a purified composition

In one or more embodiments, separating the one or more captured binding molecule-enzyme-U-polynucleotide complexes from the composition may comprise any known purification or filtering technique. In one or more embodiments, step (d) may comprise carrying out size exclusion chromatography or ion exchange purification on the composition. In one or more embodiments, the resins that are used in such processes bind to the captured binding molecule-enzyme-U-polynucleotide complexes and thereby remove them from the composition. In one or more embodiments, these techniques are applied as an alternative to the use of columns or beads.

In one or more embodiments, the composition is contacted with a capture partner for a sufficient time for the capture partner to capture the binding molecule. In one or more embodiments, a sufficient time is between 1 minute and 1 hour, preferentially between 5 minutes and 45 minutes, more preferentially between 10 minutes and 30 minutes, more preferentially around 30 minutes.

### Compositions Containing Polynucleotides

The methods and uses of the present invention are applied to compositions containing at least one polynucleotide.

In one or more embodiments, the composition containing at least one polynucleotide comprises more than one polynucleotide, preferentially it comprises a plurality of polynucleotides.

In one or more embodiments, the composition contains at least one uracil containing polynucleotide. In one or more embodiments, it may contain a plurality of uracil containing polynucleotides. In one or more embodiments, the composition containing at least one polynucleotide may contain no uracil containing polynucleotides, the methods and uses may still being applied thereto.

As described hereinabove, the composition containing at least one polynucleotide may contain any type of polynucleotide, or a mixture of different types of polynucleotide. In one or more embodiments, the composition contains DNA polynucleotides. In one or more embodiments, the composition contains ssDNA polynucleotides. In one or more embodiments, the composition containing at least one polynucleotide may contain only ssDNA polynucleotides.

In one or more embodiments, the composition containing at least one polynucleotide may be derived from or produced by any means. In one or more embodiments, the composition containing at least one polynucleotide is derived from a method of synthesising polynucleotides. In one or more embodiments, the composition containing at least one polynucleotide has been produced from a method of synthesising polynucleotides. In one or more embodiments, any method of synthesising polynucleotides may produce such a composition in which it is desirable to remove uracil containing polynucleotides. In one or more embodiments, the method of synthesising the polynucleotides is a chemical or enzymatic method. In one or more embodiments, the method of synthesising the composition containing at least one polynucleotide is an enzymatic synthesis method, preferentially a template-free enzymatic synthesis method, more preferentially an enzymatic DNA synthesis method.

In one or more embodiments, it is typical for such methods to produce compositions containing polynucleotides which contain a plurality of uracil containing polynucleotides which are undesirable. In one or more embodiments, the methods described herein enable the removal of or blockage of such uracil containing polynucleotides.

The methods of the present invention also produce compositions. In one or more embodiments, the methods of the invention produce compositions containing at least one polynucleotide. In one or more embodiments, the methods of the invention produce compositions which are purified.

In one or more embodiments, said compositions produced by the methods of the invention, which may be regarded as purified, contain fewer uracil containing polynucleotides than the composition prior to the methods being carried out, or when compared to other uracil removal processes. In one or more embodiments, fewer free uracil containing polynucleotides. By ‘free’ it is meant that the uracil containing polynucleotide is not bound to any other molecule, preferentially this does not include uracil containing polynucleotides bound to uracil DNA glycosylase enzymes.

In one or more embodiments, said compositions produced by the methods of the invention, which may be regarded as purified, contain up to 500000× fewer free uracil containing polynucleotides, 400000× fewer free uracil containing polynucleotides, 300000× fewer free uracil containing polynucleotides, 200000× free uracil containing polynucleotides, 100000× free uracil containing polynucleotides, 50000× fewer free uracil containing polynucleotides, 25000× fewer free uracil containing polynucleotides, 10000× fewer free uracil containing polynucleotides, 5000× fewer free uracil containing polynucleotides, 1000× fewer free uracil containing polynucleotides, up to 500× fewer free uracil containing polynucleotides, 400× fewer free uracil containing polynucleotides, 300× fewer free uracil containing polynucleotides, 200× fewer free uracil containing polynucleotides, 100× fewer free uracil containing polynucleotides, 50× fewer free uracil containing polynucleotides, 40× fewer free uracil containing polynucleotides, 30× fewer free uracil containing polynucleotides, 20× fewer free uracil containing polynucleotides, 10× fewer free uracil containing polynucleotides, 5× fewer free uracil containing polynucleotides, 4× fewer free uracil containing polynucleotides, 3× fewer free uracil containing polynucleotides, 2× fewer free uracil containing polynucleotides compared to compositions prior to the methods, or compared to compositions containing polynucleotides not obtained from the methods of the invention.

In one or more embodiments, the compositions produced by the methods of the invention have between 2-20× fewer free uracil containing polynucleotides, compared to compositions prior to the methods, or compared to compositions containing polynucleotides not obtained from the methods of the invention.

In one or more embodiments, the reduction in the number of free uracil containing polynucleotides in the composition may be measured by sequencing or by real-time qPCR techniques carried out on the polynucleotides produced by the process of the invention.

Sequencing and real time PCR techniques are well known in the art. In one or more embodiments, if using sequencing, one can directly determine the fraction of uracil at a particular position in a polynucleotide, and in the whole polynucleotide population before and after carrying out the processes of the invention, and compare the fractions. In one or more embodiments, if using real time qPCR, polynucleotides bound to uracil DNA glycosylase enzymes do not amplify during PCR, thus qPCR can be used to determine the fraction of uracil-free polynucleotides that remain after carrying out the processes of the invention in comparison to the total polynucleotide population before treatment.

In one or more embodiments, said compositions produced by the invention, which may be regarded as purified, contain less than 10% free uracil containing polynucleotides, less than 5% free uracil containing polynucleotides, less than 4% free uracil containing polynucleotides, less than 3% free uracil containing polynucleotides, less than 2% free uracil containing polynucleotides, less than 1% free uracil containing polynucleotides, less than 0.5% free uracil containing polynucleotides, less than 0.4% free uracil containing polynucleotides, less than 0.3% free uracil containing polynucleotides, less than 0.2% free uracil containing polynucleotides, less than 0.15% free uracil containing polynucleotides, less than 0.1% free uracil containing polynucleotides, less than 0.05% free uracil containing polynucleotides.

In one or more embodiments, the compositions produced by the invention contain less than 0.15% free uracil containing polynucleotides.

In one or more embodiments, the compositions containing at least one polynucleotide produced by the methods of the invention contain substantially no free uracil containing polynucleotides.

In one or more embodiments, the compositions containing at least one polynucleotide produced by the methods of the invention, which may be regarded as purified, may further comprise at least one uracil DNA glycosylase enzyme capable of forming a stable bond with a uracil containing polynucleotide and/or one or more uracil DNA glycosylase enzyme-U-polynucleotide complexes. In one or more embodiments, such enzymes are used in the processes of the invention and may therefore be present in the products.

In one or more embodiments, the uracil DNA glycosylase enzyme is present in the compositions produced by the methods of the invention in low amounts. In one or more embodiments, the compositions produced by the methods of the invention comprise 10 μM or less uracil DNA glycosylase enzyme.

In one or more embodiments, the compositions containing at least one polynucleotide produced by the methods of the invention relating to removal of uracil containing polynucleotides may comprise trace amounts of one or more uracil DNA glycosylase enzyme-U-polynucleotide complexes. By trace amounts it is meant less than 10 μM, preferentially less than 8 μM, more preferentially less than 6 μM, more preferentially less than 4 μM, more preferentially less than 2 μM, more preferentially less than 1 μM within said composition.

In one or more embodiments, the compositions containing at least one polynucleotide produced by the methods of the invention relating to blocking uracil containing polynucleotides comprises one or more uracil DNA glycosylase enzyme-U-polynucleotide complexes, preferentially a plurality of uracil DNA glycosylase enzyme-U-polynucleotide complexes.

### Methods of Removing or Blocking

The methods of the invention relate to either removing or blocking uracil containing polynucleotide contaminates within compositions comprising polynucleotides.

In one or more embodiments, the method of removing at least one uracil containing polynucleotide from a composition containing at least one polynucleotide comprises the steps of:
(a) Providing a composition containing at least one polynucleotide;
(b) Contacting the composition with at least one uracil DNA glycosylase enzyme, wherein each enzyme is capable of forming a stable bond with a uracil containing polynucleotide, to form one or more enzyme-U-polynucleotide complexes;
(c) Separating the one or more enzyme-U-polynucleotide complexes from the composition

In one or more embodiments, the method of blocking at least one uracil containing polynucleotide within a composition containing at least one polynucleotide comprises the steps of:

(a) Providing a composition containing at least one polynucleotide;

(b) Contacting the composition with at least one uracil DNA glycosylase enzyme, wherein the enzyme is capable of forming a stable bond with a uracil containing polynucleotide, to form one or more enzyme-U-polynucleotide complexes

In one or more embodiments, the method of blocking is a method of blocking the at least one uracil containing polynucleotide from amplification and/or sequencing. In one or more embodiments, the method of blocking is a method of preventing the at least one uracil containing polynucleotide from amplification and/or sequencing. In one or more embodiments, the method of blocking blocks amplification and/or sequencing enzymes from accessing the at least one uracil containing polynucleotide, preferentially from binding to the at least one uracil containing polynucleotide. In one or more embodiments, the method of blocking may also be termed a method of inhibiting or suppressing the detection of the at least one uracil containing polynucleotide. In one or more embodiments, the method of blocking may be termed a method of masking or silencing the at least one uracil containing polynucleotide, so that such polynucleotides are not detected. In one or more embodiments, rendering the uracil containing polynucleotide undetectable.

In one or more embodiments, step (a) of providing a composition containing at least one polynucleotide comprises synthesising a composition containing at least one polynucleotide, preferentially by enzymatic or chemical synthesis. In one or more embodiments, step (a) comprises synthesising a composition comprising at least one polynucleotide by enzymatic synthesis, preferentially template-free enzymatic synthesis. In one or more embodiments, the enzymatic synthesis is enzymatic DNA synthesis, suitably in such embodiments, the composition comprises DNA polynucleotides suitably ssDNA polynucleotides.

In one or more embodiments, step (b) of contacting the composition with at least one uracil DNA glycosylase enzyme comprises adding at least one uracil DNA glycosylase enzyme to the composition. In one or more embodiments, the amount of uracil DNA glycosylase enzyme added to the composition is equal to or greater than the amount of polynucleotides in the composition. In one or more embodiments, the uracil DNA glycosylase enzyme is added to the composition in an amount equal to or in excess relative to the amount of polynucleotides contained in the composition, preferentially in an excess relative to the amount of uracil containing polynucleotides in the composition. In one or more embodiments, the amount of uracil DNA glycosylase enzyme added to the composition is at a ratio of 1:1 or greater compared to the amount of polynucleotide in the composition. In one or more embodiments, the amount of uracil DNA glycosylase enzyme added to the composition is at a ratio of 2:1, 3:1, 4:1, or 5:1 to the amount of polynucleotide in the composition, preferentially at a ratio of 6:1, 7:1, 8:1, 9:1 or 10:1 to the amount of polynucleotide in the composition.

In one or more embodiments, the method may comprise a step of determining the amount of polynucleotides in the composition, or uracil containing polynucleotides in the composition. In one or more embodiments, prior to adding the uracil DNA glycosylase enzyme to the composition. In one or more embodiments, prior to step (b). Suitable methods of measuring the amount of polynucleotide in a composition are known in the art, for example by use of a spectrophotometer such as NanoDrop. In one or more embodiments, after the amount of polynucleotides in the composition have been determined, then the amount of required uracil DNA glycosylase enzyme can be calculated. In one or more embodiments, in accordance with the amounts indicated above.

In one or more embodiments, the method may further comprise a step of incubating the composition for a sufficient period of time to allow the uracil DNA glycosylase enzyme to bind to the uracil containing polynucleotide and form an enzyme-U-polynucleotide complex. In one or more embodiments, after step (b). Suitable periods of time for such an incubation step may be between 1 minute and 1 hour, preferentially between 5 minutes and 45 minutes, more preferentially between 10 minutes and 30 minutes, more preferentially around 30 minutes.

In one or more embodiments, step (c) of the method of removing at least one uracil containing polynucleotide which comprises separating the one or more enzyme-U-polynucleotide complexes from the composition is carried out as explained hereinabove in the binding molecule and capture partner section.

In one or more embodiments, the methods may be repeated. In one or more embodiments, the methods may be carried out more than once on the same composition. Suitably therefore steps (b) and/or (c) may be repeated, preferentially repeated one or more times. In one or more embodiments, in relation to the methods of removing at least one uracil containing polynucleotide, steps (b) and (c) are repeated, suitably the more times the method is repeated on the same composition, the more uracil containing polynucleotides that are removed, and the higher the purity of the composition obtained. In one or more embodiments, in relation to the methods of blocking at least one uracil containing polynucleotide, step (b) is repeated, suitably the more times the method is repeated on the same composition, the more uracil containing polynucleotides that are blocked.

In one or more embodiments, the method of removing at least one uracil containing polynucleotide may further comprise one or more steps of purifying the composition. In one or more embodiments, after the method has been carried out, suitably after step (b) or step (c). In one or more embodiments, after the uracil containing polynucleotides have been removed, preferentially after step (c). In one or more embodiments, the method of blocking at least one uracil containing polynucleotide may also comprise a step of purifying the composition. Suitably after step (b).

Suitable purification techniques are known in the art, such as ion exchange purification. In one or more embodiments, such purification removes other components of the composition which may have been used during the process, such as buffers, reagents or impurities.

In one or more embodiments, purification may be carried out by precipitating the polynucleotides, including the enzyme-U-polynucleotide complexes. In one or more embodiments, precipitation techniques may include contacting the composition with alcohol, such as isopropanol, preferentially to precipitate the polynucleotides, and subsequently entrapping the precipitated polynucleotides. In one or more embodiments, entrapping the precipitates may comprise contacting the precipitated polynucleotides with a surface capable of trapping said precipitated polynucleotides. In one or more embodiments, such a surface may be a porous membrane such as a silica membrane. In one or more embodiments, after the polynucleotides are entrapped in or on the surface, the surface is washed, suitably with ethanol. In one or more embodiments, the washing step allows all soluble components, such as excess UDGx enzyme and reagents, to pass through the silica membrane, but precipitated components remain entrapped. In one or more embodiments, after the washing step may be an elution step, suitably to resolubilise the polynucleotides, preferentially this is carried out with water.

In one or more embodiments, the methods may further comprise one or more steps of using the composition produced from the methods. For example, the methods may further comprise a step of amplifying the one or more polynucleotides in the composition produced from the method, preferentially after the method has been carried out, preferentially after step (b) or step (c). In one or more embodiments, therefore only desirable polynucleotides are amplified, preferentially substantially no uracil containing polynucleotides are amplified. In one or more embodiments, the one or more polynucleotides in the composition may be amplified by PCR.

In one or more embodiments, the methods may further comprise a step of sequencing the one or more polynucleotides in the composition produced from the methods preferentially after the method has been carried out, preferentially after step (b) or step (c). In one or more embodiments, therefore only desirable polynucleotides are sequenced, preferentially substantially no uracil containing polynucleotides are sequenced. In one or more embodiments, the one or more polynucleotides in the composition may be sequenced by NGS.

In one or more embodiments, the methods may further comprise a step of amplifying the one or more polynucleotides in the composition and a subsequent step of sequencing the one or more polynucleotides in the composition.

### Methods of Polynucleotide Synthesis

The methods of the invention further encompass methods of synthesising polynucleotides which comprise a step of adding a uracil DNA glycosylase enzyme capable of forming a stable bond with at least one uracil containing polynucleotide into the reaction mixture or composition.

Furthermore, as described above, the methods of removing or blocking uracil containing polynucleotide may be carried out on compositions obtained from methods of synthesising polynucleotides.

Suitably the method of polynucleotide synthesis is enzymatic or chemical. Suitably enzymatic polynucleotide synthesis, suitably template-free enzymatic synthesis. In one or more embodiments, it is enzymatic DNA synthesis, in such an embodiment, preferentially the polynucleotides are DNA, preferentially the polynucleotides are ssDNA.

In one or more embodiments, the methods may further comprise one or more steps of synthesising a composition comprising at least one polynucleotide. Suitably the methods may further comprise one or more steps of synthesising a composition comprising at least one polynucleotide by enzymatic synthesis. In one or more embodiments, the methods may further comprise one or more steps of synthesising a composition comprising at least one polynucleotide by template-free enzymatic synthesis. In one or more embodiments, the methods may further comprise one or more steps of synthesising a composition comprising at least one DNA polynucleotide by enzymatic DNA synthesis.

In one or more embodiments, the method of removing at least one uracil containing polynucleotide from a composition containing at least one polynucleotide comprises the steps of:
(a) Synthesising a composition containing at least one polynucleotide by enzymatic polynucleotide synthesis;
(b) Contacting the composition with at least one uracil DNA glycosylase enzyme wherein the enzyme is capable of forming a stable bond with a uracil containing polynucleotide to form one or more enzyme-U-polynucleotide complexes;
(c) Separating the one or more enzyme-U-polynucleotide complexes from the composition

In one or more embodiments, the method of blocking at least one uracil containing polynucleotide within a composition containing at least one polynucleotide comprises the steps of:
(a) Synthesising a composition containing at least one polynucleotide by enzymatic polynucleotide synthesis;
(b) Contacting the composition with at least one uracil DNA glycosylase enzyme wherein the enzyme is capable of forming a stable bond with a uracil containing polynucleotide to form one or more enzyme-U-polynucleotide complexes

In one or more embodiments, the one or more steps of enzymatic polynucleotide synthesis may be carried out as described in WO2020/165137 for example, suitably such methods may be template-free. In one or more embodiments, the steps are demonstrated in FIG. 1 which may be incorporated herein. In one or more embodiments, the one or more steps of enzymatic polynucleotide synthesis may comprise: (a) providing an initiator nucleic acid having a free 3′hydroxyl group (b) contacting the initiator nucleic acid, or an elongated nucleic acid thereof, with a protected nucleotide and a polymerase such that the nucleic acid is elongated by incorporation of the protected nucleotide, (c) deprotecting the protected nucleotide of the elongated nucleic acid, and (d) repeating steps (b) and (c) until the polynucleotide is formed.

In one or more embodiments, the protected nucleotide may comprise a 3′-O-protected nucleotide. Guidance in selecting 3′-O-protecting groups and corresponding deprotecting conditions for the above method may be found in the following references: U.S. Pat. Nos. 5,808,045; 8,808,988; International patent publication WO9 1/06678. Suitably, a deprotection agent is used to deprotect the protected nucleotide in step (c). Suitably a deprotection agent is a chemical cleaving agent, such as, for example, dithiothreitol (DTT). Alternatively, a deprotection agent may be an enzymatic deprotection agent, such as, for example, a phosphatase, which may cleave a 3′-phosphate protecting group. It will be understood by the person skilled in the art that the selection of the deprotection agent depends on the type of 3′-nucleotide protection group used, whether one or multiple protection groups are being used, whether initiator nucleic acids are attached to living cells or organisms or to solid supports, and the like, that necessitate mild treatment. For example, a phosphine, such as tris(2-carboxyethyl)phosphine (TCEP) can be used to deprotect a 3′O-azidom ethyl groups, palladium complexes can be used to deprotect a 3′O-allyl groups, or sodium nitrite can be used to deprotect a 3′O-amino group. In some embodiments, the deprotection step involves TCEP, a palladium complex or sodium nitrite.

In one or more embodiments, it is desirable to employ two or more different protecting groups that may be removed using orthogonal deprotection conditions. The following exemplary pairs of protecting groups may be used in parallel synthesis embodiments in which two or more polynucleotide sequences are synthesised in the same reaction mixture. It is understood that other deprotecting group pairs, or groups containing more than two, may be available for use.

TABLE 2
Protecting Group Pairs

| | |
|---|---|
| 3′-O—NH2 | 3′-O azidomethyl |
| 3′-O—NH2 | 3′-O-allyl, 3′O-propargyl |
| 3′-O—NH2 | 3′-O-phosphate |
| 3′-O-azidomethyl | 3′-O-allyl, 3′O-propargyl |
| 3′-O-azidomethyl | 3′-O-phosphate |
| 3′-O-allyl, 3′O-propargyl | 3′-O-phosphate |

In one or more embodiments, if the polynucleotide to be synthesised is RNA then the protected nucleotide is an rNTP (ribonucleoside triphosphate). In one or more embodiments, the elongation may comprise between 125-500 μM protected rNTP. Suitable protected rNTPs may be 3′-O-blocked rNTPs. In one or more embodiments, the rNTP may be a 3′-O-azidomethyl-rNTP. In one or more embodiments, which may be selected from protected A, C, G and U ribonucleosides. In one or more embodiments, which may be selected from 3′-azidomethyl-O-adenosine triphosphate, 3′-azidomethyl-O-guanosine triphosphate, 3′-azidomethyl-O-cytidine triphosphate, and 3′-azidomethyl-O-uridine triphosphate. Alternatively, the 3′-blocked nucleotide triphosphate is blocked by either 3′-0-propargyl, a 3′-0-azidomethyl, 3′-0-NH₂ 3′-0-allyl group, 3′-0-methyl, 3′-0-(2-nitrobenzyl), 3′-0-allyl, 3′-0-amine, 3′-0-azidomethyl, 3′-0-tert-butoxy ethoxy, 3′-0-(2-cyanoethyl), or 3′-0-propargyl group. Suitably, any of the 3′-0-blocked rNTPs employed in the invention may be purchased from commercial vendors (e.g. Jena Bioscience, MyChemLabs, or the like) or synthesized using published techniques, e.g. U.S. Pat. No. 7,057,026; International patent publications WO2004/005667, WO91/06678; Canard et al, Gene (cited above); Metzker et al, Nucleic Acids Research, 22:4259-4267 (1994); Meng et al, J. Org. Chem., 14:3248-3252 (3006); U.S. patent publication 2005/037991; Zavgorodny et al, Tetrahedron Letters, 32(51): 7593-7596 (1991).

In one or more embodiments, the polymerase is a template-free polymerase. Suitably a template-free DNA polymerase. Suitable template-free polymerases are known in the art, in some embodiments, the template-free DNA polymerase is a terminal deoxynucleotidyl transferase (TdT) or modified form thereof such as those described in WO2020/099451. In one or more embodiments, the polymerase may be a poly(A) polymerase or a poly(U)polymerase.

In one or more embodiments, exemplary reaction conditions for elongation step (b) of the enzymatic synthesis method may comprise the following: 2.0 pM purified TdT; 125-600 pM 3′-O-blocked dNTP (e.g. 3′-O—NHi-blocked dNTP); about 10 to about 500 mM potassium cacodylate buffer (pH between 6.5 and 7.5) and from about 0.01 to about 10 mM of a divalent cation (e.g. CoCU or MnCU), where the elongation reaction may be carried out in a 50 pL reaction volume, at a temperature within the range RT to 45° C., for 3 minutes.

In one or more embodiments, during the elongation step, the nucleic acid is elongated by incorporation of a protected nucleotide, suitably an individual protected nucleotide. In one or more embodiments, in each cycle of steps (b) to (d) of the enzymatic synthesis method, an individual protected nucleotide is added to the nucleic acid. In one or more embodiments, the protected nucleotide to be added is determined by the sequence of the polynucleotide to be synthesised.

In one or more embodiments in which the 3′-O-blocked dNTPs are 3′-O—NH2-blocked dNTPs, exemplary reaction conditions for deprotecting step (c) of the enzymatic synthesis method may comprise the following: 700 mM NaNCE; 1 M sodium acetate (adjusted with acetic acid to pH in the range of 4.8-6.5), where the deprotecting reaction may be carried out in a 50 pL volume, at a temperature within the range of RT to 45° C. for 30 seconds to several minutes.

In one or more embodiments, after the deprotecting step, the deprotected elongated nucleic acid comprises a free 3′-hydroxyl group for the cycle of steps (b) to (d) of the synthesis to be repeated to add further nucleotides to the elongated nucleic acid.

In one or more embodiments, an “initiator nucleic acid” refers to a short oligonucleotide sequence with a free 3′hydroxyl group, which can be further elongated by a polymerase, suitably a template-free polymerase, such as TdT. In one or more embodiments, the initiator nucleic acid is DNA. In one or more embodiments, the initiator nucleic acid is RNA. Suitably the initiator nucleic acid comprises between 3 and 100 nucleotides, in particular between 3 and 20 nucleotides. In one or more embodiments, the initiator nucleic acid is single-stranded. In an alternative embodiment, the initiator nucleic acid is double-stranded.

In one or more embodiments, the initiator nucleic acid is provided on a surface, suitably the initiator nucleic acid is tethered to a surface, suitably a solid surface. In one or more embodiments, the initiator nucleic acid is tethered to the surface at its 5′ end, and comprises a free 3′hydroxyl. In one or more embodiments, the surface may be an inert material such as a sepharose, agarose resin, or a particle or bead, suitably in some embodiments, the bead may be magnetic. In a particular embodiment, an initiator nucleic acid synthesized with a 5′-primary amine may be covalently linked to magnetic beads using the manufacturer's protocol. Likewise, an initiator nucleic acid synthesized with a 3′-primary amine may be covalently linked to magnetic beads or agarose beads using the manufacturer's protocol. A variety of other attachment chemistries amenable for use with the invention are well-known in the art, e.g. Integrated DNA Technologies brochure, “Strategies for Attaching Oligonucleotides to Solid Supports,” v.6 (2014); Hermanson, Bioconjugate Techniques, Second Edition (Academic Press, 2008).

In one or more embodiments, the initiator nucleic acid may comprise a primer, suitably a primer at its free end, which may be its 3′ end. In one or more embodiments, the primer is used by the polymerase to begin polymerisation of the polynucleotide. A suitable primer may be a poly rNTP sequence such as a poly rATP sequence. In one or more embodiments, the initiator nucleic acid comprises a primer of (rATP) 5.

In one or more embodiments, the method may further comprise a step of cleaving the completed polynucleotide from the initiator nucleic acid. Suitably after step (d). In one or more embodiments, if the elongated nucleic acid after step (b) is complete, then a final deprotection step (c) occurs and the polynucleotide is cleaved from the initiator nucleic acid. Suitably by use of an endonuclease enzyme. In one or more embodiments, by use of an EndoV enzyme. In one or more embodiments, the initiator nucleic acid may suitably comprise an internal cleavage point, in this case a deoxyinosine residue. In one or more embodiments, the cleavable inosine residue is penultimate to the 3′-terminal nucleotide of the initiator nucleic acid. In one or more embodiments, a cleavable linker may be used in the initiator nucleic acid. Further means of cleaving polynucleotides are disclosed in U.S. Pat. Nos. 5,739,386, 5,700,642, P5830655 for example.

In one or more embodiments, cleavage may leave a 5-hydroxyl on the polynucleotide product, or may leave a moiety at the 5′ end. In one or more embodiments, the method may comprise a further step of removing 5′ moieties from the polynucleotide, for example by phosphatase treatment.

In one or more embodiments, the method may comprise one or more wash steps, suitably between each round of repetition. In one or more embodiments, the wash steps are carried out with wash buffer. In one or more embodiments, the wash steps remove any unused nucleotides. In one or more embodiments, there is at least a first wash after the elongation step and a second wash after the deprotection step.

In one or more embodiments, the above methods may further comprise a step of capping the polynucleotide. In one or more embodiments, after elongation and deprotection steps. In one or more embodiments, a capping step may be included in which a free 3′-hydroxyl is reacted with a compound that prevents any further elongation of the capped strand. In one or more embodiments, such a compound may be a dideoxy nucleoside triphosphate.

In one or more embodiments, the step of adding the uracil DNA glycosylase enzyme takes place at any stage in the method of polynucleotide synthesis. In one or more embodiments, adding the uracil DNA glycosylase enzyme takes place after the or each polynucleotide has been synthesised, preferentially after the composition comprising at least one polynucleotide is synthesised. In one or more embodiments, after step (d) of the synthesis process described above. In one or more embodiments, during clean-up of the polynucleotide or the composition containing the polynucleotide.

In one or more embodiments, the method of polynucleotide synthesis may comprise the steps of:
(a) providing one or more initiator nucleic acids having a free 3′hydroxyl end;
(b) contacting the or each initiator nucleic acid, or one or more elongated nucleic acids, with a protected nucleotide and a polymerase such that the or each nucleic acid is elongated by incorporation of the protected nucleotide;
(c) deprotecting the protected nucleotide of the or each elongated nucleic acid;
(d) repeating steps (b) and (c) until at least one polynucleotides is formed;
(e) optionally cleaving the or each polynucleotide from the initiator nucleic acid to form a composition containing at least one polynucleotide; and
(f) adding a uracil DNA glycosylase enzyme which is capable of forming a stable bond with at least one uracil containing polynucleotide to the composition, suitably to form at least one enzyme-U-polynucleotide complex if a uracil containing polynucleotide is present

In one or more embodiments, the uracil DNA glycosylase enzyme is added to remove or to block at least one uracil containing polynucleotide, preferentially which may have been produced during the synthesis. In one or more embodiments, the steps of the methods of removing or blocking uracil containing polynucleotides as described herein may be incorporated into the method of polynucleotide synthesis. In one or more embodiments, the method may comprise one or more further steps as described in relation to the methods of removing or blocking uracil containing polynucleotides hereinabove.

In one or more embodiments, the method may comprise a further step of separating one or more enzyme-U-polynucleotide complexes, preferentially after the step of adding the uracil DNA glycosylase enzyme. In one or more embodiments, any method of separation may be used, such as the use of binding molecules and capture partners as described hereinabove.

In one or more embodiments, the method of polynucleotide synthesis may comprise the steps of:
(a) providing one or more initiator nucleic acid having a free 3′hydroxyl end;
(b) contacting the or each initiator nucleic acid, or one or more elongated nucleic acids, with a protected nucleotide and a polymerase such that the or each nucleic acid is elongated by incorporation of the protected nucleotide;
(c) deprotecting the protected nucleotide of the or each elongated nucleic acid;
(d) repeating steps (b) and (c) until at least one polynucleotide is formed;
(e) optionally cleaving the or each polynucleotide from the initiator nucleic acid to form a composition containing at least one polynucleotide;
(f) adding at least one uracil DNA glycosylase enzyme to the composition, wherein the enzyme is capable of forming a stable bond with a uracil containing polynucleotide, suitably to form at least one enzyme-U-polynucleotide complex if a uracil containing polynucleotide is present; and
(g) separating the or each enzyme-U-polynucleotide complex if present

### Methods of Tethering

The present invention further relates to methods and uses of uracil DNA glycosylase enzymes to tether uracil containing polynucleotides to surfaces or molecules.

The strong bond created when a uracil DNA glycosylase enzyme described herein binds to a uracil containing polynucleotide can be used as a means to attach polynucleotides to surfaces or to other molecules.

In one or more embodiments, a surface may comprise one or more uracil DNA glycosylase enzyme-U-polynucleotide complexes tethered thereto. Suitably therefore a molecule may comprise one or more uracil DNA glycosylase enzyme-U-polynucleotide complexes tethered thereto. In one or more embodiments, such a molecule may be termed a ‘complex’ comprising a uracil DNA glycosylase enzyme attached to a molecule, wherein the uracil DNA glycosylase enzyme is further stably bound to a uracil containing polynucleotide.

In one or more embodiments, in any case, the uracil DNA glycosylase enzyme is stably bound to the uracil containing polynucleotide.

In one or more embodiments, each molecule may comprise more than one uracil DNA glycosylase enzyme attached thereto, and more than one uracil containing polynucleotide stably bound thereto.

In one or more embodiments, the surface may be any solid surface, suitably any inert solid surface. Suitably the surface may be a plate, slide, array, column, bead, fabric, mesh, or the like. Alternatively the surface may be a liquid such as a gel.

In one or more embodiments, the molecule may be any biological molecule. Suitably any biological molecule such as a protein or a nucleic acid. Suitably a therapeutic or diagnostic biological molecule, or a biological molecule which may be used as a reagent. Suitably a therapeutic or diagnostic protein, or a protein reagent. Suitable proteins may be antibodies or antigen binding fragments thereof, aptamers, peptides, TCRs, CARs, enzymes and the like.

In one or more embodiments, the molecule is an enzyme, suitably an enzyme which cleaves nucleic acids, suitably an enzyme which cleaves DNA such as an endonuclease. In one or more embodiments, the enzyme is EndoV. Suitably EndoV is capable of cleaving DNA. Suitably such a complex may be used to target the enzyme to the vicinity of a particular uracil base in a polynucleotide for activity on the polynucleotide at a target position, such as cleavage at a particular position. Suitably the uracil base may be placed at the target position in the polynucleotide, suitably prior to contact with the complex.

In one or more embodiments, the uracil containing polynucleotide may also have a specific function. Suitably the uracil containing polynucleotide may be a therapeutic agent, or a reagent. Suitably the uracil containing polynucleotide may be a therapeutic nucleic acid, such as for example an antisense oligonucleotide, an miRNA, an siRNA, an ssiRNA, an RNAi, a splice switching oligonucleotide, a gapmer, a mixmer, or the like. Suitably the uracil containing polynucleotide may be a reagent, such as for example, it may comprise a barcode for use in labelling or subsequent sequencing.

In one or more embodiments, the molecule is an antibody or antigen binding fragment thereof and the uracil containing polynucleotide comprises a sequencing barcode. In one or more embodiments, such a complex may be used to perform NGS-based analysis of antibody binding.

In one or more embodiments, the molecule may be used to target a therapeutic nucleic acid to the correct site. In one or more embodiments, the molecule may be an antibody or antigen binding fragment thereof or aptamer and the uracil containing polynucleotide may be a therapeutic nucleic acid. In such a way, the complexes of the present invention may be used as drug conjugates, for example antibody drug conjugates.

In one or more embodiments, the molecule described herein may equally be referred to as a ‘molecule of interest’.

In one or more embodiments, the method of tethering at least one polynucleotide to a surface comprises the steps of:
(a) Depositing one or more uracil DNA glycosylase enzymes on or in a surface, wherein each uracil DNA glycosylase enzyme is capable of forming a stable bond with a uracil containing polynucleotide;
(b) Contacting the surface with at least one uracil containing polynucleotide to form one or more enzyme-U-polynucleotide complexes

In one or more embodiments, step (a) of depositing the enzymes onto or into the surface may comprise attaching the enzymes to the surface via a linker for example, via a binding tag and capture partner in a similar manner to that described hereinabove, or via chemical conjugation. Suitable means of depositing the enzyme are as follows.

In one or more embodiments, step (a) may comprise adsorbing the enzymes onto the surface, suitably by hydrophobic interactions and salt linkages. In one or more embodiments, step (a) may comprise bonding the enzymes to the surface, preferably by covalent bonding. In one or more embodiments, the covalent bonding may be between one or more amino acid side chains in the enzyme to one or more functional groups on the surface such as imidazole, indolyl, phenolic groups, hydroxyl groups, for example. In one or more embodiments, the surface may be activated, and may comprise one or more cyanogen bromide groups which covalently bind to the enzyme. In one or more embodiments, step (a) may comprise affinity immobilisation, preferably wherein the surface comprises an affinity ligand, and the enzyme is attached to an affinity partner which binds to the affinity ligand, such as the SpyCatcher-SpyTag system. Alternatively still, in embodiments where the surface is a gel or fabric, step (a) may comprise entrapping or embedding the enzyme within the surface.

In one or more embodiments, the method may further comprise a step of adding one or more uracil nucleotides to at least one polynucleotide, suitably prior to step (b). Suitably to form at least one uracil containing polynucleotide, which may be used in step (b). Suitably the uracil is added to the 5′ or the 3′ end of the or each polynucleotide. Suitably the uracil is ligated to the 5′ or the 3′ end of the or each polynucleotide. Suitably therefore the uracil DNA glycosylase enzyme binds to the 5′ or 3′ end of the polynucleotide.

In one or more embodiments, the uracil nucleotide may be incorporated into a polynucleotide during polynucleotide synthesis, which may be enzymatic or chemical synthesis as described above. Alternatively, the uracil nucleotide may be incorporated into a polynucleotide during amplification, for example during PCR amplification. Alternatively still, the uracil nucleotide may be created in a polynucleotide by chemical methods, such as deamination of a cytosine base to a uracil base, suitably after synthesis of the polynucleotide or amplification thereof.

In one or more embodiments, the method may further comprise a step of incubating the surface with the at least one uracil containing polynucleotide for a sufficient time for the or each uracil DNA glycosylase enzyme to bind to the or each uracil containing polynucleotide. Suitably after step (c). Suitable periods of time for such an incubation step may be between 1 minute and 1 hour, suitably between 5 minutes and 45 minutes, suitably between 10 minutes and 30 minutes, suitably around 30 minutes.

In one or more embodiments, the method of tethering a polynucleotide to a molecule of interest comprises the steps of:
(a) Attaching a uracil DNA glycosylase enzyme to a molecule to form an enzyme-molecule complex, wherein the uracil DNA glycosylase enzyme is capable of forming a stable bond with a uracil containing polynucleotide;
(b) Contacting the enzyme-molecule complex with a uracil containing polynucleotide to allow the uracil DNA glycosylase enzyme to bind to the uracil containing polynucleotide.

In one or more embodiments, step (a) may comprise attaching the uracil DNA glycosylase enzyme to a molecule via a linker for example. Suitably the uracil DNA glycosylase enzyme may be fused to a molecule, suitably therefore the uracil DNA glycosylase enzyme may be expressed as a fusion protein with the molecule. Suitably the fusion protein may comprise a linker located between the uracil DNA glycosylase enzyme and the molecule.

In one or more embodiments, the method may further comprise a step of adding one or more uracil nucleotides to at least one polynucleotide, suitably prior to step (b). Suitably to form at least one uracil containing polynucleotide, which may be used in step (b). Suitably the uracil is added to the 5′ or the 3′ end of the or each polynucleotide. Suitably the uracil is ligated to the 5′ or the 3′ end of the or each polynucleotide. Suitably therefore the uracil DNA glycosylase enzyme binds to the 5′ or 3′ end of the polynucleotide.

In one or more embodiments, the method may further comprise a step of incubating the enzyme-molecule complex with the at least one uracil containing polynucleotide for a sufficient time for the or each uracil DNA glycosylase enzyme to bind to the or each uracil containing polynucleotide. Suitably after step (c). Suitable periods of time for such an incubation step may be between 1 minute and 1 hour, suitably between 5 minutes and 45 minutes, suitably between 10 minutes and 30 minutes, suitably around 30 minutes.

### Nucleic Acids and Host Cells

The invention further provides nucleic acids encoding uracil DNA glycosylase enzymes of the invention as defined hereinabove, and corresponding vectors comprising the nucleic acids and host cells comprising either the proteins or the nucleic acids.

In one or more embodiments, there is provided a nucleic acid molecule comprising a nucleotide sequence which encodes a uracil DNA glycosylase enzyme of the invention.

Suitably, the invention also encompasses nucleic acids which hybridize, under stringent conditions, to a nucleic acid encoding uracil DNA glycosylase enzymes of the invention as defined above. In one or more embodiments, such stringent conditions include incubations of hybridization filters at about 42° C. for about 2.5 hours in 2×SSC/0.1% SDS, followed by washing of the filters four times of 15 minutes in 1×SSC/0.1% SDS at 65° C. Protocols used are described in such reference as Sambrook et al. (Molecular Cloning: a Laboratory Manual, Cold Spring Harbor Press, Cold Spring Harbor N.Y. (1988)) and Ausubel (Current Protocols in Molecular Biology (1989)).

Optionally the nucleic acid molecule may be isolated.

An “isolated” nucleic acid molecule is substantially separated away from other nucleic acid sequences with which the nucleic acid is normally associated, such as, from the chromosomal or extrachromosomal DNA of a cell in which the nucleic acid naturally occurs. A nucleic acid molecule may be an isolated nucleic acid molecule when it comprises a transgene or part of a transgene present in the genome of another organism. The term also embraces nucleic acids that are biochemically purified so as to substantially remove contaminating nucleic acids and other cellular components. Isolated nucleic acids are substantially free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. The isolated nucleic acid molecule may be flanked by its native genomic sequences that control its expression in the cell, for example, the native promoter, or native 3 ‘untranslated region.

In one or more embodiments, the nucleic acid molecule may be comprised upon a vector, suitably an expression vector. The term “vector” refers to DNA molecule used as a vehicle to transfer recombinant genetic material into a host cell. The major types of vectors are plasmids, bacteriophages, viruses, cosmids, and artificial chromosomes. The vector itself is generally a DNA sequence that consists of an insert (a heterologous nucleic acid sequence, transgene) and a larger sequence that serves as the “backbone” of the vector. The purpose of a vector which transfers genetic information to the host is typically to isolate, multiply, or express the insert in the target cell.

In one or more embodiments, the vector can be either RNA or DNA, either prokaryotic or eukaryotic, and typically is a virus or a plasmid. In one or more embodiments, the vector is a plasmid. Suitable vectors include any vector for bacterial expression, such as any pET expression vector.

In one or more embodiments, the expression vector may further comprise one or more regulatory elements to aid expression of the nucleic acid molecule. The term “regulatory element” or “regulatory sequence” as used herein refers to a nucleic acid that is capable of regulating the transcription and/or translation of an operably linked nucleic acid molecule. Regulatory elements include, but are not limited to, promoters, enhancers, introns, 5′ UTRs, and 3′ UTRs. For example, the expression vector may contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal. Such a portion of an expression vector may be referred to as an expression cassette.

“Expression cassette” as used herein means a nucleic acid sequence capable of directing expression of a particular nucleic acid sequence in an appropriate host cell, comprising a promoter operably linked to the nucleic acid sequence, in this case a nucleic acid molecule comprising a sequence encoding a uracil DNA glycosylase enzyme, which is operably linked to termination signal sequences. It also typically comprises sequences required for proper translation of the nucleic acid sequence. The expression cassette comprising the nucleic acid sequence may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular nucleic acid sequence of the expression cassette does not occur naturally in the host cell. The expression of the nucleic acid molecule in the expression cassette may be under the control of, for example, a constitutive promoter or of an inducible promoter that initiates transcription only when the host cell is exposed to some particular external stimulus.

Expression cassettes may include in the 5 ‘-3’ direction of transcription, a transcriptional and translational initiation region (e.g., a promoter), a nucleic acid molecule comprising a sequence encoding a uracil DNA glycosylase of the invention, and a transcriptional and translational termination region (e.g., termination region).

In one or more embodiments, the expression vector or expression cassette may comprise in the 5 ‘-3’ direction of transcription, a 5′UTR, a promoter, a nucleic acid molecule comprising a sequence encoding a uracil DNA glycosylase of the invention, and a 3′UTR. Suitably the 5′UTR, the promoter and the nucleic acid are operably linked.

Any promoter can be used in the production of the expression cassettes and vectors including such expression cassettes as described herein. The promoter may be native or analogous, or foreign or heterologous, to the host and/or to the nucleic acid sequence. Additionally, the promoter may be a natural sequence or alternatively a synthetic sequence. Where the promoter is “foreign” or “heterologous” to the host, it is intended that the promoter is not found in the native host into which the promoter is introduced. Where the promoter is “foreign” or “heterologous” to the nucleic acid molecule, it is intended that the promoter is not the native or naturally occurring promoter for the operably linked nucleic acid molecule. Any promoter can be used in the preparation of expression cassettes to control the expression of the nucleic acid molecule. In one or more embodiments, the promoter is the native promoter of the uracil DNA glycosylase, suitably of the wild type uracil DNA glycosylase from which the modified enzyme is derived.

The expression cassettes may also comprise transcription termination regions. Where transcription terminations regions are used, any termination region may be used in the preparation of the expression cassettes. For example, the termination region may be native to the transcriptional initiation region, may be native to the operably linked nucleic acid molecule, may be native to the host, or may be derived from another source (i.e., foreign or heterologous to the promoter, the nucleic acid molecule of the invention, the host, or any combination thereof).

In addition, other sequence modifications can be made to the nucleic acid molecules of the invention. For example, additional sequence modifications that are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

In preparing the expression cassettes and expression vectors described herein, the various nucleic acid molecules may be manipulated, so as to provide for the nucleic acid molecules in the proper orientation and, as appropriate, in the proper reading frame. Toward this end, adapters or linkers may be employed to join the nucleic acid molecules or other manipulations may be involved to provide for convenient restriction sites, removal of superfluous nucleic acid molecules, removal of restriction sites, or the like. For this purpose, in vitro mutagenesis, primer repair, restriction, annealing, resubstitutions, e.g., transitions and transversions, may be involved.

Expression vectors may include additional features. For example, they may include additional features such as selectable markers, e.g. Phosphomannose Isomerase (PMI), and antibiotic resistance genes that can be used to aid recovery of stably transformed hosts.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence or nucleic acid molecule that is operably linked to a second nucleotide sequence or nucleic acid molecule, means a situation when the first nucleotide sequence or nucleic acid molecule is placed in a functional relationship with the second nucleotide sequence or nucleic acid molecule. For instance, a promoter is operably associated with a nucleotide sequence or nucleic acid molecule if the promoter effects the transcription or expression of said nucleotide sequence or nucleic acid molecule. Those skilled in the art will appreciate that the control sequences (e.g., promoter) need not be contiguous with the nucleotide sequence or nucleic acid molecule to which it is operably associated, as long as the control sequences function to direct the expression thereof. Thus, for example, intervening untranslated, yet transcribed, sequences can be present between a promoter and a nucleotide sequence or nucleic acid molecule, and the promoter can still be considered “operably linked” to or “operatively associated” with the nucleotide sequence or nucleic acid molecule.

In one or more embodiments, the uracil DNA glycosylase enzymes of the invention may be produced by industrial fermentation of host cells engineered to express and produce the uracil DNA glycosylase enzymes.

The term “expression”, as used herein, refers to any step involved in the production of a polypeptide including, but not being limited to, transcription, post-transcriptional modification, translation, post-translational modification, and secretion.

In one or more embodiments, the host cell of the invention may be any cell from any organism. Suitably the host cell may be a prokaryotic or eukaryotic cell. Suitable the host cell may be a bacterial, fungal, plant, insect or animal cell. In one or more embodiments, the host cell is a bacterial cell. In one or more embodiments, the bacterial cell may be any bacterial cell typically used for industrial fermentation process, for the production of proteins on an industrial scale.

In one or more embodiments, the host cell is an E. coli cell. Suitably an E. coli cell selected from E. coli BL21 (DE3).

In one or more embodiments, the host cell may be transformed, transfected or transduced in a transient or stable manner with a nucleic acid of the invention, encoding a uracil DNA glycosylase enzyme. In one or more embodiments, the host cell may be transformed, transfected or transduced in a transient or stable manner with a vector or expression cassette which comprises a nucleic acid of the invention encoding a uracil DNA glycosylase enzyme. Suitable means of transforming, transfecting or transducing a host cell are well known in the art.

The nucleic acid, expression cassette or expression vector according to the invention may be introduced into the host cell by any method known by the skilled person, such as electroporation, conjugation, transduction, competent cell transformation, protoplast transformation, protoplast fusion, biolistic “gene gun” transformation, PEG-mediated transformation, lipid-assisted transformation or transfection, chemically mediated transfection, lithium acetate-mediated transformation, liposome-mediated transformation.

In one or more embodiments, more than one copy of a nucleic acid, cassette or vector of the present invention may be inserted into a host cell to increase production of the uracil DNA glycosylase enzyme.

In one or more embodiments, the host cell may be cultured, suitably in a fermentation medium, suitably in an industrial fermentation process, under suitable conditions to express a nucleic acid encoding a uracil DNA glycosylase enzyme of the invention. Suitable such culture conditions and suitable mediums for the production of proteins are well known in the art.

In one or more embodiments, the host cells may secrete the uracil DNA glycosylase enzymes into the fermentation medium. Alternatively, the host cells may be lysed to release the uracil DNA glycosylase enzymes into the fermentation medium. In one or more embodiments, the uracil DNA glycosylase enzymes are recovered from the fermentation medium ready for use in the methods described herein.

### Kit

The invention further provides a kit comprising a uracil DNA glycosylase enzyme of the invention as defined hereinabove, and one or more reagents for carrying out a process of the invention, suitably for carrying out a process of blocking or removing uracil containing polynucleotides according to the invention.

In one or more embodiments, the kit may further comprise a binding molecule and a capture partner, as defined hereinabove. In one or more embodiments, the binding molecule may already be attached to the uracil DNA glycosylase enzyme or may be provided separately for attachment to the uracil DNA glycosylase enzyme. Optionally the capture partner may be free or may be immobilised upon a substrate such as a column or bead. In one or more embodiments, the kit may comprise a substrate such as a column or bead comprising one or more capture partners.

In one or more embodiments, the uracil DNA glycosylase enzyme in the kit may be immobilised upon a substrate. In one or more embodiments, therefore the kit may simply comprise a substrate comprising the uracil DNA glycosylase immobilised thereon. Suitably in such embodiments, no capture partner is included in the kit.

In one or more embodiments, an ion exchange or size exclusion chromatography resin or cartridge, or a silica membrane may be provided in the kit. In one or more embodiments, one or more purification reagents may be provided in the kit, such as an alcohol, such as isopropanol. In one or more embodiments, in addition to the binding molecule and capture partner. In one or more embodiments, for purification of the polynucleotides.

In one or more embodiments, the kit may comprise reagents for separation of the enzyme-U-polynucleotide complexes from the composition. In one or more embodiments, a desalting buffer, eluting buffer, wash buffer and the like may be present in the kit.

In one or more embodiments, the kit may further comprise one or more reagents for synthesis, amplification, and/or sequencing of a polynucleotide.

In one or more embodiments, the reagents may comprise one or more primers or initiator nucleic acids, or adapters. In one or more embodiments, one or more pairs of primers. Suitably the initiator nucleic acids may be comprised on a support, for example an array or slide. In one or more embodiments, the initiator nucleic acids may each comprise a free 3′hydroxyl group. In one or more embodiments, the initiator nucleic acids may each further comprise a cleavable site, for example a restriction site, an inosine cleavable nucleotide, or a photocleavable linker. In one or more embodiments, initiator nucleic acids are defined hereinabove.

In one or more embodiments, the reagents may comprise one or more nucleotides. Suitably one or more A, U, C, T, G nucleotides. In one or more embodiments, the nucleotides may be dNTPs or rNTPs, suitably dNTPs such as dA, dU, dC, dT, or dG. In one or more embodiments, the one or more nucleotides may be protected nucleotides, suitable for template-free enzymatic synthesis of polynucleotides. In one or more embodiments, the one or more nucleotides may be 3-O-protected nucleotides such as 3′-O-amino-dNTPs or 3′-O-azidomethyl-dNTPs. Suitable the reagents may comprise a mixture of nucleotides, preferentially a mixture in the required ratios of each nucleotide for the polynucleotide to be synthesised.

In one or more embodiments, the reagents may comprise one or more enzymes. Suitably a polymerase enzyme, suitably an RNA or DNA polymerase. In one or more embodiments, a polyA or polyU polymerase. In one or more embodiments, a Taq polymerase. Suitably a terminal deoxynucleotidyl transferase (TdT) enzyme. In one or more embodiments, the reagents may further comprise an endonuclease enzyme capable of cleaving polynucleotides.

Suitably a DNA endonuclease. In one or more embodiments, EndoV. In one or more embodiments, the endonuclease is for cleaving the polynucleotide from the solid support, if present.

In one or more embodiments, the reagents for carrying out a process of the invention or for synthesis, amplification, and/or sequencing may further comprise one or more buffers, salts, stabilisers, chelating agents, dyes and the like. In one or more embodiments, the reagents may further comprise an elongation buffer for carrying out elongation of the polynucleotide sequence, a deprotection buffer for deprotecting the protected nucleotides, if present. In one or more embodiments, the reagents may comprise wash buffer for washing away unreacted nucleotides, or unbound uracil containing polynucleotides.

In one or more embodiment, there is provided a kit comprising a uracil DNA glycosylase enzyme of the invention as defined hereinabove, and one or more reagents for blocking or removing uracil containing polynucleotides, and one or more reagents for synthesis of a polynucleotide, suitably for enzymatic synthesis of a polynucleotide, suitably for template-free enzymatic synthesis of a polynucleotide. In such an embodiment suitably the kit comprises a uracil DNA glycosylase enzyme of the invention, one or more of the components for removing the uracil containing polynucleotides as described above, an initiator nucleic acid on a solid support, one or more protected nucleotides, a terminal deoxynucleotidyl transferase (TdT) enzyme or other suitable polymerase enzyme, an elongation buffer, a deprotection buffer, a wash buffer, an endonuclease enzyme such as EndoV, and a cleavage buffer. In one or more embodiments, the uracil DNA glycosylase enzyme is provided together with the endonuclease enzyme, suitably in a storage buffer.

In an aspect of the invention there is provided a composition comprising a uracil DNA glycosylase enzyme as defined herein, combined with an endonuclease enzyme. In one or more embodiments, the endonuclease enzyme may be EndoV. In one or more embodiments, in a storage buffer. In one or more embodiments, the composition is for carrying out a method of the invention.

In one or more embodiments, the components of the kit may be comprised within suitable packaging, suitably containers. In one or more embodiments, the kit may further comprise instructions for use.

### DESCRIPTION OF THE FIGURES

The invention will now be described with reference to the following figures in which:

FIG. 1 shows: Analysis of different UDGx enzyme activity on IDT oligo containing U base on SDS-PAGE gel. With the exception of UDGx5 and 10, all UDGx enzymes bind FAM-U-oligo with differing extent.

FIG. 2 shows: Effects of the ancestral UDGx6 enzyme activity on IDT HER2 and HER2-U template on qPCR reaction. The amount of DNA template for HER2-U with UDGx6 treatment is approximately 100 000 times lower than HER2-U without UDGx6 treatment.

FIG. 3A shows: The position of the deoxyuracil, indicated by X, in each primer set as well as the predicted PCR outcome.

FIG. 3B shows: Demonstrates the ability of primers, with/without deoxyuracil at the 5′, centre or 3′ regions, and treated or untreated with ancestral UDGx6, to prime a qPCR reaction. Failure to amplify the template DNA, or a reduction in template copy number, is indicative of reduced functionality of the primers. In the absence of UDGx, the efficacy of the primers was only minorly affected the presence of a uracil. In the presence of UDGx, primer efficacy was either reduced (5′ end or centre) or completely abolished (3′ end), depending on the location of the uracil.

FIG. 4 shows The number of reads from sequencing (NGS) analysis for IDTq37 (SEQ ID NO 27) and IDTq37t (SEQ ID NO 26). The number of reads for no UDGx treatment (standard) shows that it corresponds well to the expected ratio between IDTq37: IDTq37t. The UDGx treated samples do not have any reads for IDTq37t, which suggests that the UDGx enzymes bind to IDTq37t, leading to absence of oligo products in NGS run.

FIG. 5 shows: Overall sequencing (NGS) results showing the % of C to U substitution error for enzymatically synthesised oligos treated with the ancestral UDGx6, UDGx7 or UDGx8 enzymes, or without.

FIG. 6 shows: More specific sequencing (NGS) results showing the % of C to U C to T substitution error for individual enzymatically synthesised oligos when treated with the ancestral UDGx6, 7 or 8 enzymes or without.

FIG. 7 shows: Overall sequencing (NGS) results showing the % of C to U substitution error for q21 and q37 enzymatically synthesised oligos treated with 20 U or 40 U USER (comparative process) or without USER.

### EXAMPLES

The invention will now be described with reference to the following non-limiting examples:

### Materials and Methods

### Synthetic UDGx Constructs

Putative protein sequences encoding UDGx-like enzymes were sourced from the NCBI database using the keyword search and BLAST functions. Ancestral sequences were derived from selected UDGx-like sequences using MEGA the software (https://www.megasoftware.net/). HIS-Tag and Strep-Tactin binding Tag amino acid sequences were added to the N- and C-terminals of each selected sequence, respectively. The final polypeptide sequences (SEQ IDs 1 to 5, 6, 8 and 10) were back-translated to DNA and codon optimized for expression in Escherichia coli, synthesized and cloned into pET28a (+) by Eurogentec (https://www.eurogentec.com/).

### Expression and Purification

The pET28a (+) vectors containing the synthetic UDGx-encoding genes were transformed into E. Cloni® EXPRESS BL21 (DE3) (Lucigen) as per manufacturer's instructions. Single colonies were inoculated into 6 ml 2× YT media (Sigma-Aldrich) supplemented with kanamycin (50 μg·ml⁻¹) and 0.5% glucose and incubated overnight at 37° C. with shaking at 200 RPM in an Innova shaking incubator. Two milliliters of each starter culture was inoculated into 500 ml fresh 2× YT media in an Erlenmeyer flask and supplemented with kanamycin and glucose as before. The cultures were incubated at 37° C. with shaking at 200 RPM until the optical density, measured at 600 nm, reached ˜1.0. Thereafter the growing cultures were transferred to a 20° C. shaking incubator where it was incubated for 30 min prior to induction with 0.5 mM IPTG and supplemented with FeCl3 50 μM. After ˜16 hours of expression, the cells were collected by centrifugation in an Eppendorf Centrifuge 5920 R at 3428× g for 40 minutes. The spent media was discarded and the cell pellets stored at −80° C. until needed.

To extract and purify the recombinant UDGx proteins, the cell pellets were lysed in the presence of 22.5 ml lysis buffer (25 mM TRIS pH 8.5, 0.3% Triton 100, 1 mM CaCl₂), 1 μl·ml-1 DNAse I, 0.2 mg·ml-1 lysozyme, 1 mM DTT) for 2 h at 25° C. with vigorous shaking. Thereafter, 2.5 ml of a 5 M NaCl was added and incubated for an additional hour at 25° C. with vigorous shaking. The cell debris and insoluble protein was separated from the soluble protein by centrifugation at 3428×g for 40 min at 15° C. The lysate was transferred into a new 50 ml conical tube and the His-tagged UDGx proteins were captured using 100 μl equilibrated HisPur™ Ni-NTA Resin (ThermoScientific). After binding for 30 min, the entire mixture was filtered through a Bio-Spin Disposable Chromatography Column (Bio-Rad). Unwanted protein, salts and cellular components were washed away by step-wise applying 50 ml wash buffer (25 mM TRIS-CI pH 8.5, 10 mM imidazole, 0.5 M NaCl) to the column over a custom vacuum unit set to 600 mBar. The purified UDGx was eluted from the Ni-NTA resin by applying 200 μl elution solution (25 mM TRIS-CI pH 8.5, 300 mM imidazole, 0.5 M NaCl) and collecting the flow-through in a 1.5 ml microfuge tube by means of centrifugation at 2000×g in an Eppendorf microfuge. Finally, glycerol was added to a final concentration of 35% for storage at −20° C.

### Gel Shift Assays

To demonstrate the binding activity of the UDGx enzymes, 10 μM of enzyme was combined with 20 μM single stranded oligonucleotide substrate with/without deoxyuracil (SEQ ID 14 and 15, respectively) in 0.5 mM TRIS-HCL pH 8.5, 1 mM EDTA and 5 mM β-mercaptoethanol in a 20 μl reaction. The reactions were incubated at 37° C. for 30 min to allow binding. Thereafter, 20 μl of Laemmli buffer (Bio-Rad) was added to each sample and subjected to thermal denaturation at 95° C. for 5 min. Ten microliters of each denatured protein sample were loaded on a 4-15% Criterion™ TGX Stain-Free™ Protein Gel (Bio-Rad) set in a Criterion™ Cell (Bio-Rad) and separated according to size by electrophoresis at 200V for 40 min in 1×TGX buffer. After electrophoresis, the gels were stained with InstantBlue and visualized on an Gel Doc™ EZ Imager (Bio-Rad). The images were analyzed using Image Lab software (Bio-Rad).

### Real-Time PCR Quantification

To determine whether UDGx-bound oligonucleotides can serve as a template during PCR amplification, UDGx, at concentrations ranging from 0.75 to 12 μM, was combined with 7.5 uM oligonucleotide without deoxyuracil (SEQ ID 15) or with four deoxyuracils (SEQ ID 16) in a buffer containing suitable concentrations of NaCl, MgCl2 and TRIS HCl PH 8.0. A control reaction without UDGx was included in the set. After incubation at 37° C. for 30 min, the samples were diluted to 0.004 nM in molecular biology grade MilliQ H₂O. Five microliters of each reaction were used as template in a qPCR assay designed to specifically quantify the deoxyuracil-containing DNA. In addition to the template DNA, the qPCR reaction contained 1 μM each forward (SEQ ID 18) and reverse primer (SEQ ID 19) and 1×iTaq Supermix SYBR Green (Bio-Rad) in a 20 μl reaction. A standard curve, consisting of the oligonucleotide template spanning 4 nM to 4.0×10-⁵ nM, was included to extrapolate the amount or deoxyuracil-containing template in the treated and control samples. The CFX96 qPCR instrument was programmed to denature the DNA at 95° C. and thereafter cycle through 44 cycles of denaturation at 95° C. for 30 sec, anneal and extend at 60° C. for 30 sec before executing a melting curve from 65 to 95° C. The amplification signal was captured at the end of every cycle. The qPCR data was analysed using the Bio-Rad CFX Maestro software.

To determine whether UDGx-bound oligonucleotide can prime a PCR reaction, a similar approach was used except that here the primers (SEQ ID 19 to 24) instead of the template contained deoxyuracil, and the template (SEQ ID 15) was pure rather than a mixture of DNA. The reagent concentrations and cycling parameters were similar to those described above.

### UDGx to Improve Purity

To demonstrate that UDGx was capable of binding and removing low abundance deoxyuracil-containing sequences from an oligonucleotide solution, deoxyuracil-containing (Seq ID 25) and deoxyuracil-free (SEQ ID 26) oligonucleotides were combined in a 1:10 ratio to a final concentration of 7.5 μM mixed oligonucleotide in combination with 10 μM of either modified UDGx6, 7 or 8 in 170 mM NaCl, 50 mM MgCl2 and 10 mM TRIS HCl pH 8.0. The reactions were incubated for 30 min at 37° C., whereafter the oligonucleotides were purified from the enzyme and salt mixtures. Briefly, the oligonucleotides were precipitated by the addition of 3 volumes of 100% isopropanol and transferred to a Binding Plate E (Invitec). The isopropanol was subsequently removed by vacuum filtration, and the remaining salt and other impurities were washed away by two rounds of vacuum filtration of 80% EtOH (800 μl). After drying, the purified polynucleotides were eluted by twice adding 50 μl molecular biology grade H₂O and collection by centrifugation in an Eppendorf Centrifuge 5920 R at 3428× g for 5 minutes.

To determine the fraction of deoxyuracil-containing sequences after each treatment, the purified oligos were prepared for sequencing on an iSeq 100 System (Illumina) using an xGen™ ssDNA & Low-Input DNA Library Preparation Kit (IDT) using 7.5 pmol DNA input per sample. The libraries were quantified on a Qubit using the 1×dsDNA HS kit (Invitrogen) and loaded onto the sequencing flow cell at 50 pM. The sequencing reads were mapped to a reference sequence and the C to U deamination frequency was determined using a custom in-house pipeline. In short, forward and reverse FASTQ files were processed through the following packages: FastQC for sequencing quality control, Trimmomatic to trim Illumina adapters, BBMerge to merge forward and reverse reads, Bowtie 2 to locally align the reads against the expected sequence and generate BAM alignment files and finally bam-readcount for variant calling.

To demonstrate that UDGx would mediate a decrease in C to U deaminations in enzymatically synthesized oligonucleotides by eliminating deoxyuracil-containing sequences, 24 different sequences, each 52 nucleotides in length, were synthesized using an in house, proprietary technology. Briefly, the oligonucleotide sequences were synthesized at 750 pmol scale on a bead-based solid support. The synthesis process consisted of 52 rounds of Terminal deoxynucleotidyl transferase (TdT) mediated incorporation of ONH₂-blocked nucleotides, followed by chemical deblocking of the ONH₂ group using suitable concentrations of TdT enzyme such as 1 μM in 500 mM Cacodylate Buffer with 10 mM CoCl₂, nucleotides such as 500 μM and a suitable deblocking agent such as sodium nitrite buffer (pH 5) at a concentration of 1 M, and two rounds of washing before starting the next cycle using a suitable wash buffer such as 500 mM LiCl with 0.01% Tween-20 and 10 mM ETDA. The synthesized oligonucleotides were liberated from the resin using 0.36 μM EndoV to cleave the oligonucleotides at a designated deoxyinosine in Liberation buffer for 30 min at 37° C. The released DNA was collected in a collection plate by centrifugation in an Eppendorf Centrifuge 5920 R at 3428×g for 5 min. The liberated oligonucleotides were purified from the enzyme and salt mixture through desalting on a Binding Plate E, prepared for sequencing on an iSeq 100 System and analyzed as described above.

### Results

### 1) UDGx Constructs

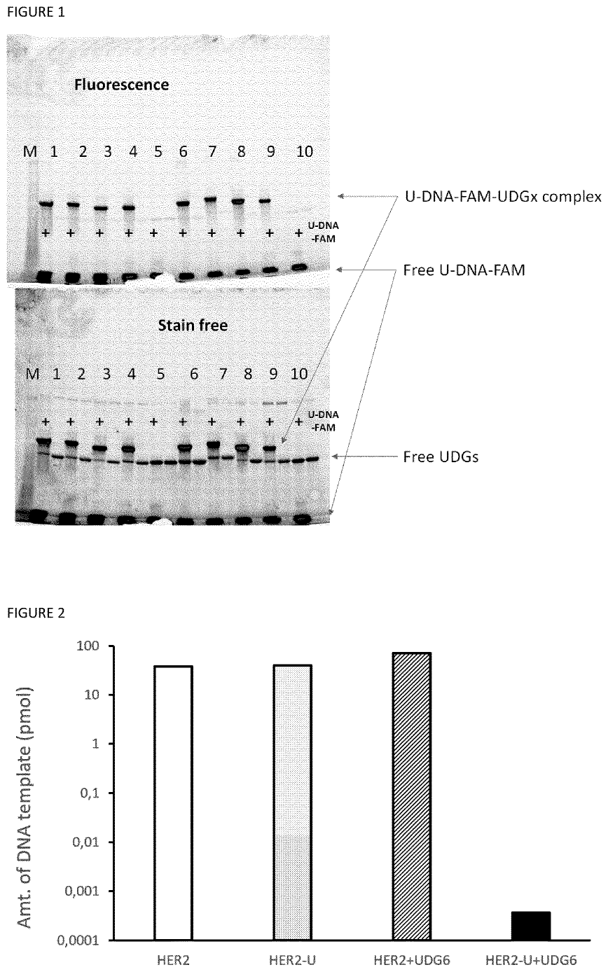

The sequence of 7 wild type UDGx genes were sourced from the NCBI database (Table 1, SEQ ID NOs 1 to 5, 11 and 12). Additionally, an ancestral sequence was derived for the UDGx sequences of Mycolicibacterium themoresistible (SEQ ID NO: 6), Mycobacterium colombiense (SEQ ID NO: 8) and Rhodochrous rhodochrous (SEQ ID NO: 10) using the MEGA software (https://www.megasoftware.net/). Finally, a N-terminal HIS-Tag and C-terminal Strep-Tactin binding Tag was added for protein purification on Ni-NTA resin and optional removal of UDGx from enzymatic reactions with Streptactin-Sepharose (IBA) resin, respectively. All 10 UDGx enzymes numbered 1-10 were well expressed, purified and, with the exception of UDGx5 and 10, capable of covalently binding deoxyuracil-containing DNA as demonstrated in FIG. 1. Here, the purified UDGx enzymes were incubated with either a deoxyuracil-containing (SEQ ID NO: 13 FAM-U-oligo) or -free (SEQ ID NO: 14 TAM-oligo) oligonucleotide and the products analyzed on SDS PAGE gel. FAM is a fluorescent dye corresponding to a single isomer derivative of fluorescein which is been abbreviated 56-FAM when used in nucleotide sequences, and attached at the 5′ end of the nucleotide. TAM corresponds to 5′ TAMRA (NHS ester), which is abbreviated 56-TAMN when used in nucleotide sequences, and attached to the 5′ end of the nucleotide. An upwards size shift was observed for the UDGx enzymes when it was co-incubated with deoxyuracil-containing oligonucleotide, but not with deoxyuracil-free oligonucleotide. Furthermore, the UDGx-oligonucleotide linkage remained even after heat inactivation of the enzyme, thereby demonstrating that the linkage between enzyme and oligonucleotide was covalent.

TABLE 1
UDGx constructs name and number as used in the examples.

| SEQ | UDGx | | |
|---|---|---|---|
| ID NO | number | Name | Info |
| 1 | 1 | Mycolicibacterium | |
| | | smegmatis | |
| 2 | 2 | Mycolicibacterium | |
| | | thermoresistibile | |
| 3 | 3 | Gandjariella thermophila | |
| 4 | 4 | Rhodocococcus ruber | |
| 5 | 5 | Human_mut_145_268 | |
| 6 | 6 | Ancestral modified 182-anc | 86% ID to UDGx |
| | | | from |
| | | | Mycolicibacterium |
| | | | thermoresistibile |
| | | | (SEQ ID NO: 2) |
| 8 | 7 | Ancestral modified 6-anc | 91% ID to UDGx |
| | | | from Mycobacterium |
| | | | colombiense (SEQ |
| | | | ID NO: 7) |
| 10 | 8 | Ancestral modified 179-anc | 87% ID to UDGx |
| | | | from Rhodococcus |
| | | | rhodochrous (SEQ |
| | | | ID NO: 9) |
| 11 | 9 | Amycolatopsis viridis | |
| 12 | 10 | Bovine mut | |

2) Analysis of IDT Oligo Products Treated with or without UDGx6 on qPCR

To demonstrate that binding of UDGx to an uracil-containing oligo would prevent that oligo from serving as a template in a PCR reaction, thereby preventing C to U deaminations in PCR amplification, for example, the ancestral UDGx6 enzyme was incubated in the presence of two different oligonucleotide templates: One, HER2 (SEQ ID No: 15), is identical to a region of the human HER gene whereas the other, HER2-U (SEQ ID No: 16), has four of the deoxycytosine bases replaced with deoxyuracil. The UDGx-DNA mixtures were then diluted in water and the amount of amplifiable template DNA was determined using an HER2-specific quantitative Real-Time PCR assay. In the absence of ancestral UDGx6, the four deoxyuracils in HER2-U did not affect amplification as the amount of HER2-U template DNA quantified was similar to the HER2 control (FIG. 2). However, treatment of the HER2-U template with UDGx6 reduced the amplifiable template copy number by more than 100,000-fold while the template copy number of the HER2 control was not reduced by UDGx6. Therefore, UDGx specifically bound the deoxyuracil bases and rendered those oligonucleotides no longer suitable as tenplate for PCR amplification.

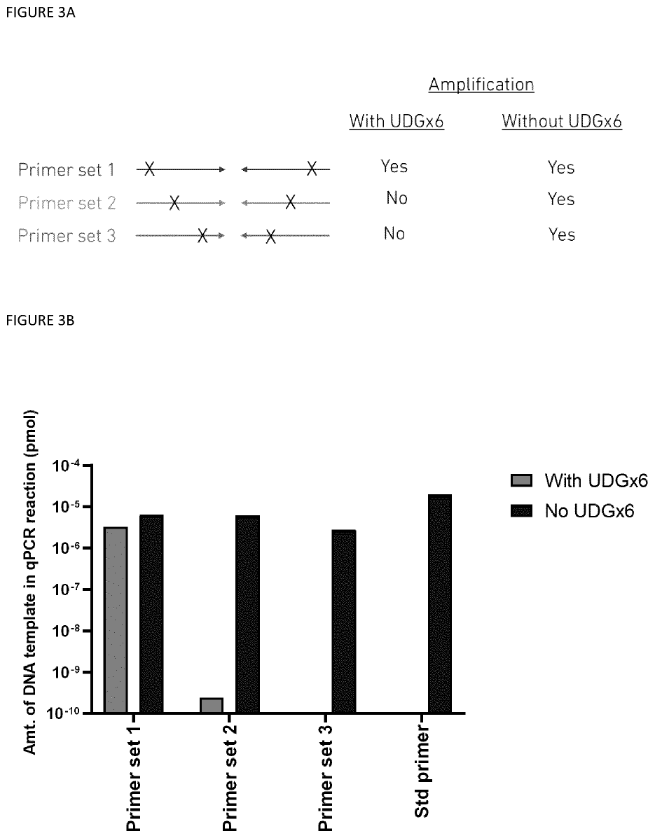

To demonstrate that UDGx-bound oligonucleotides would not prime PCR amplification, four pairs of deoxyuracil-containing primers, where a deoxyuracil base was either absent (SEQ ID NOs 17 and 18) or close to the 5′ end (SEQ ID NOs 19 and 20), center (SEQ ID NOs 21 and 22) or 3′ end (SEQ ID NOs 23 and 24) of the primer, were tested for their ability to prime a quantitative PCR reaction with or without ancestral UDGx6 enzyme bound to the primers. In the absence of the ancestral UDGx6, all deoxyuracil-containing primers were able to prime the qPCR reaction. The amount of template DNA quantified in these control reactions was 3 to 9-fold lower compared the deoxyuracil-free control primer, depending on whether the deoxyuracil was position near the 5′, middle or 3′ ends of each primer (FIG. 3). When ancestral UDGx6 was bound near the 5′ end, the amount of template DNA quantified was 3-fold lower than when UDGx6 was absent, suggesting that binding of UDGx to the 5′ side of a primer only minorly affected it's priming function. When ancestral UDGx6 was bound to a deoxyuracil located in the center of the primers, the amount of template DNA quantified was >108-fold lower than the corresponding deoxyuracil-free control. Lastly, when the UDGx was bound to a deoxyuracil near the 3′ end of the primers, the primers were no longer capable of priming the PCR amplification. Therefore, these results demonstrate that UDGx can be applied to prevent/limit the interaction of deoxyuracil-containing oligonucleotides in biological applications such as PCR.

3) Analysis of IDT Oligo Products Treated with or without UDGx on NGS

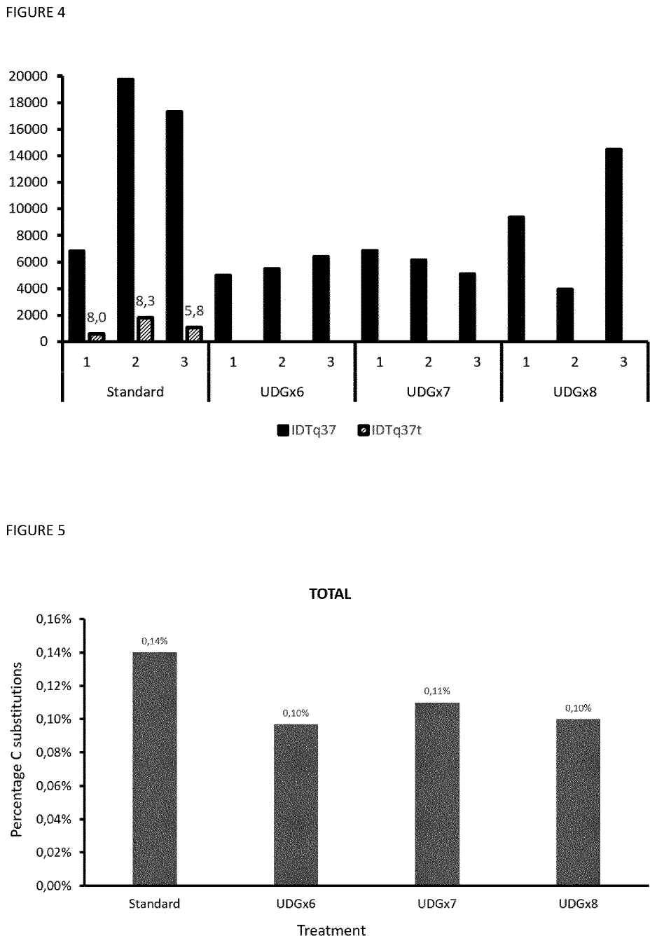

To demonstrate that UDGx enzymes were capable of removing uracil-containing oligos occurring at a low frequency within an oligonucleotide population, deoxyuracil-containing (SEQ ID NO: 25 IDTq37t) and -free (SEQ ID NO:26 IDTq37) oligonucleotides were combined 1 in 10, respectively, and incubated with or without UDGx6, UDGx7 and UGDx8 enzymes (ancestral sequences). The fraction of deoxyuracil-containing oligonucleotides present after treatment and purification of the oligonucleotide mixtures were determined from the number of NGS reads for each sequence in the mixture. In the absence of UDGx treatment, the number of deoxyuracil-containing reads were 6 to 8% (FIG. 4). However, when treated with either of the three UDGx enzymes, the number of deoxyuracil-containing reads decreased to below detectable levels (<1 in 1000 reads or 0.1%). These results therefore demonstrate the ability of UDGx to remove deoxyuracil-containing oligonucleotides from an oligonucleotide pool.

4) Analysis of EDS Oligo Products Treated with or without UDGx on NGS

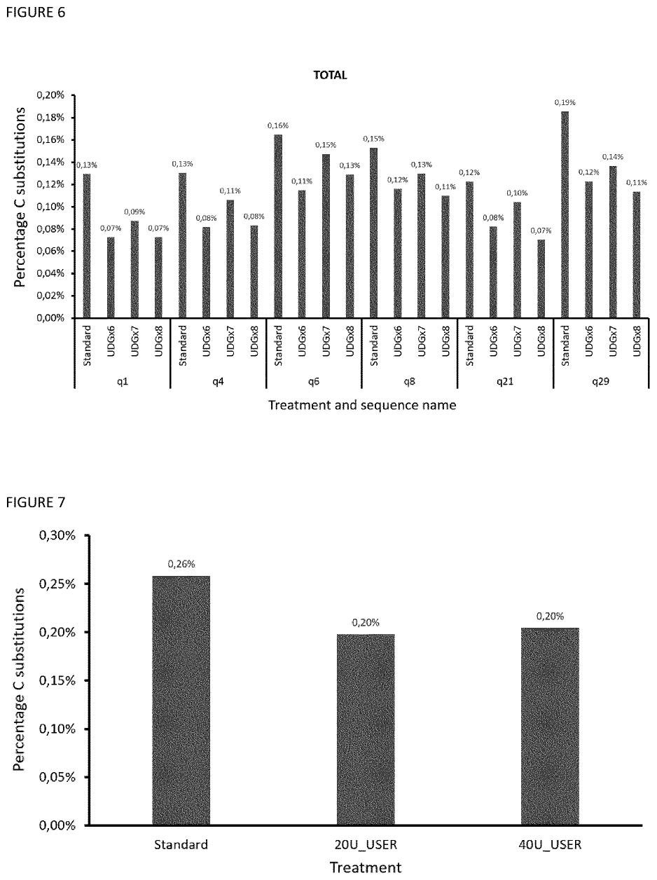

Enzymatic DNA synthesis suffers from increased deamination of deoxycytosine to deoxyuracil, which manifests as C to U deamination. To determine if UDGx enzymes would reduce the apparent C to U deamination error rate following EDS synthesis, 6 different oligonucleotides (SEQ ID NOs 27 to 32) were synthesized in-house and treated in the presence or absence of three different UDGx enzymes (UDGx6, UDGx7 and UDGx8). Analysis of the C to U transition rates observed in the NGS data revealed that the average C to U transition rate across all 6 oligonucleotides was 0.14% (FIG. 5). It is to be noted that in NGS, the U will appear as T because it is the complement of A. Treatment of these oligos with either of the three UDGx enzymes reduced the C to U transition rate by ˜20 to ˜30%. While the UDGx-mediated decrease in C to U transition rate was variable between different sequences and for different UDGx enzymes, a decrease was nonetheless observed in all 6 sequences (FIG. 6).

Enzyme cocktails, such as Uracil-Specific Excision Reagent (USER), developed to destroy uracil-containing oligonucleotides, are commercially available. USER is a mixture of an UDG enzyme, which excises the uracil base from an oligonucleotide to create an abasic site, and an endonuclease VIII enzyme, which cleaves the phosphodiester backbone to create two separate oligonucleotide molecules. In a control experiment to demonstrate the maximum decrease in C to U transition error rate that could be achieved on EDS oligonucleotides using a commercially available enzyme cocktail, oligonucleotide q21 (SEQ ID NO 28) and q37 (SEQ ID NO 33), were synthesized in house and treated without or with 20, 40 Units of USER in the supplied buffer prior to liberation from the solid support. Analysis of the C to U transition rate following NGS revealed that, irrespective of whether 20 or 40 Units of USER was used, the average C to U transition rate was ˜23% lower for the USER-treated samples than the non-treated control samples (FIG. 7). Given that UDGx reduced C to U transitions by 20 to 30%, depending on the sequence, the results demonstrate that UDGx, which is a single enzyme rather than mixture of enzymes, is at least equally as effective as a commercially available enzyme cocktail such as USER at eliminating deoxyuracil-containing oligonucleotides from an EDS oligonucleotide pool.

In conclusion, it was demonstrated that these UDGx enzymes bind covalently to deoxyuracil-containing oligonucleotides, thereby rendering them inactive as template or primer in PCR applications, for example, or to mediate a reduction in the apparent C to U transition error rate that is otherwise exaggerated in enzymatic DNA synthesis.

### SEQUENCES

SEQ ID NO: 1 UDGx1 from
Mycobacterium smegmatis
MGAQDFVPHTADLAELAAAAGECRGCGLYRDATQAVFGAGGRSARIMMIG
EQPGDKEDLAGLPFVGPAGRLLDRALEAADIDRDALYVTNAVKHFKFTRA
AGGKRRIHKTPSRTEVVACRPWLIAEMTSVEPDVVVLLGATAAKALLGND
FRVTQHRGEVLHVDDVPGDPALVATVHPSSLLRGPKEERESAFAGLVDDL
RVAADVRP
SEQ ID NO: 2 UDGx2 from
Mycolicibacterium thermoresistibile
MAVTGAARFVPATRDLGELAEAVHACKGCDLYVDATQAVFGAGPGTAPMV
MVGEQPGDREDTAGQPFVGPAGRLLQRALDAAGIDRAEVYVTNAVKHFTF
TRGRRLIHKTPSRSDVVACRPWLIAELDSVRPEVVVLLGATAAKSLLGPD
FRLTAHRGEVLRLPAGDATVGLGVDPLVVVTVHPSAVLRGRPGDRAEAFD
ALVADLEVAAGLMGS
SEQ ID NO: 3 UDGx3 from
Gandjariella thermophila
MGVAQRQSAAPFVPSGAGIDELREAASGCRGCSLYRDATQTVFGQGSPRA
RLMMIGEQPGDREDRQGAPFVGPAGRLLNRALEEAGLPRDSVYLTNAVKH
FKFERVHGKQRIHKKPSRTEVVACWPWLAAELGVVRPEFAVCLGATAAQA
LLGTSFRVTAHRGELLDGPRYADDADPLVVVATVHPSSVLRAPDPEARED
AYRRLVADLGVAASAMAGW
SEQ ID NO: 4 UDGx4 from
Rhodococcus ruber
MAVSRQPGAEEFVPDSTDLAELAAAAGDCRGCELYRNAERTVFGAGPASA
RLVLVGEQPGDQEDRAGEPFVGPAGRLLDRALEEAGIDRDEVYVTNAVKH
FKFERAAAGGRRIHKKPARGEIVACRPWLVAELQAVRPDVLVCLGATAAQ
SLLGPSFRVTAHRGEILHPDVRVPSDPAVVATIHPSAILRGPSAQREEAL
AGLVADLRVAAGVL
SEQ ID NO: 5 UDGx5 from
Homo sapiens
MEFFGESWKKHLSGEFGKPYFIKLMGFVAEERKHYTVYPPPHQVFTWTQM
CDIKDVKVVILGQNPYHGPNQAHGLCFSVQRPVPPPPSLENIYKELSTDI
EDFVHPGHGDLSGWAKQGVLLLNAVLTVRAHQANSHKERGWEQFTDAVVS
WLNQNSNGLVFLLWGSYAQKKGSAIDRKRHHVLQTANPSPLSVYRGFFGC
RHFSKTNELLQKSGKKPIDWKEL
SEQ ID NO: 6 UDGx6 modified (synthetic)
‘182-anc’ (derived from UDGx2 of
SEQ ID NO: 2)
MAVTGAARFVPETRDLGELAEAAHGCKGCDLYRDATQTVFGAGPGSAPMM
LVGEQPGDQEDRAGQPFVGPAGRLLDRALEEAGIDRAQVYVTNAVKHFKF
TRGKRRIHKTPSRTEVVACRPWLIAELDSVRPEVVVCLGATAAQSLLGPD
FRVTAHRGEVLRLPAEDATVTVDVDPRVVVTVHPSAVLRGRPEDRAEAFD
ALVADLRVAAGLM
SEQ ID NO: 7 UDGx7 from
Mycobacterium colombiense
MAATSNAPGAARYLPEERGLDALRDAAETCHGCSLFEDATQTVFGNGHPG
APIMLVGEQPGDQEDRAGEPFVGPAGRLLDRALEDAGIDPAMVYETNAVK
HFKFTRKGGKRRIHQKPGRTEVVACRPWLIAEIEAVRPQVIVCLGATAAQ
SLLGATFRVSTQRGQQLRLPSTVDVHLTPEPTLVATVHPSSVLRDRSDRH
DEVYEAFVDDLRSAGAGLGRSG
SEQ ID NO: 8 UDGx7 modified (synthetic)
‘6-anc’ (derived from UDGx7 of
SEQ ID NO: 7)
MAATSNAPGADRYLPEERDLDALRDAAETCRGCSLFEDATQTVFGNGHPG
APIMLVGEQPGDQEDRAGEPFVGPAGRLLDRALEDAGIDPALVYVTNAVK
HFKFTRKGGKRRIHQKPGRTEVVACRPWLIAEIEAVRPEVIVCLGATAAQ
SLLGSDFRVSAQRGQQLRLPASVDVDLAPEPTVVATVHPSSVLRDRSDRH
DEAYQSFVDDLRSAGGGL
SEQ ID NO: 9 UDGx8 from
Rhodococcus rhodochrous
MAVSRQPGAGEFVPETTDLAELAAAASGCRGCDLYRNAERTVFGAGPATA
RLVLVGEQPGDQEDRAGEPFVGPAGRLLDRALEEAGIDRGEVYVTNAVKH
FKFERAAAGGRRIHKKPARGEVVACRPWLVAELQAVRPEVLVCLGATAAQ
SLLGPSFRVTAHRGEILHPDAEIPSDPAVVATIHPSAILRGPSEQREEAL
AGLVADLRVAAGAL
SEQ ID NO: 10 UDGx8 modified (synthetic)
‘179-anc’ (derived from UDGx8 of SEQ
ID NO: 9)
MAVSRQPGAAEFVPETRDLAELAAAARGCRGCDLYRDATQTVFGAGPATA
RMMLVGEQPGDQEDRAGEPFVGPAGRLLDRALEEAGIDRERVYVTNAVKH
FKFTRAEGGKRRIHKKPSRTEVVACRPWLVAELQAVRPEVLVCLGATAAQ
SLLGPSFRVTAHRGEVLHLPAEVESDPRVVATVHPSAVLRGPSEDRDEAF
AALVADLRVAAGAL
SEQ ID NO: 11 UDGx9 from
Amycolatopsis viridis
MATSTRRDAAEFVPDSRSLDRLRSAALRCQGCDLHRDATQTVFGDGPAPA
KVLMLGEQPGDKEDVAGEPFVGPAGRLLDRALDEAGVDRSQVYVTNAVKH
FKFVRGERGKQRIHKKPSRGEIVACRPWLVAELEAVQPSVVVLLGATAAS
SLMGPSFRVTAHRGELLPAPEEFAGRPERVLATVHPSSVLRAPDRDEAYA
ALVADLRSVPEMM
SEQ ID NO: 12 UDGx10 from Bovine mut
MIFGESWKKHLSGEFGKPYFIKLMGFVAEERKHYTVYPPPHQVFTWTQMC
DIRDVKVVILGQNPYHGPNQAHGLCFSVQRPVPPPPSLENIYKELSTDVD
GFVHPGHGDLSGWAKQGVLLLNAVLTVRAHQANSHKERGWEQFTDAVVSW
LNQNAHGLVFLLWGSYAQKKGSAIDRKRHHVLQTANPSPLSVYRGFFGCR
HFSKTNELLQKSGKEPINWKDL
SEQ ID NO: 13 synthetic FAM-U-oligo:
/56-FAM/TTTTTTT/ideoxyU/TTTTTTT
SEQ ID NO: 14 synthetic TAM-oligo:
/56-TAMN/TTTTTTTTTTTTTTT
SEQ ID NO: 15 HER2
ACGGACGTGGGATCCTGCACCCTCGTCTGCCCCCTGCACAACCAAGAGGT
GACAGCAGAGGATGGAACACAGCGGTGTGAGAAG
SEQ ID NO: 16 synthetic HER2-U
ACGGACGTGGGATCCTGCACC/ideoxyU/TCGT/ideoxyU/TGCC/
ideoxyU/CCTG/ideoxyU/ACAACCAAGAGGTGACAGCAGAGGAT
GGAACACAGCGGTGTGAGAAG
SEQ ID NO 17: Fwd control
ACGGACGTGGGATCCTGCA
SEQ ID NO 18: Rev control
CTTCTCACACCGCTGTGTTCCAT
SEQ ID NO 19: 5′ Fwd
A/ideoxyU/GGACGTGGGATCCTGCA
SEQ ID NO 20: 5′ Rev
CTTCTCACACCGCTGTGTTC/ideoxyU/AT
SEQ ID NO 21: Center-Fwd
ACGGA/ideoxyU/GTGGGATCCTGCA
SEQ ID NO 22: Center-Rev
CTTCTCACAC/ideoxyU/GCTGTGTTCCAT
SEQ ID NO 23: 3′-Fwd
ACGGACGTGGGATCCTG/ideoxyU/A
SEQ ID NO 24: 3′-Rev
CTT/ideoxyU/TCACACCGCTGTGTTCCAT
SEQ ID NO: 25 IDTq37t:
TTACAGATGATGG/ideoxyU/GGTT/ideoxyU/AGG/ideoxyU/A/i
deoxyU/A/ideoxyU/AGGGGT/ideoxyU//ideoxyU/GTG/ideox
yU/GGTT/ideoxyU//ideoxyU/G/ideoxyU/A/ideoxyU/AGGG/
ideoxyU/AATGG
SEQ ID NO: 26 IDTq37:
TTACTAGTGATGGCGGTTCAGGCACACAGGGGTCCGTGCGGTTCCGCACA
GGGCAATGG
SEQ ID NO: 27 q1
ACGACCTACAGAACAAACCGGGGTTCCGAGCGGTAATAGCAACACCAACG
GG
SEQ ID NO: 28 q21
GTATGGCGCGATGACTCGCGCACGCTACGGATTCACTTGCTAAATATCAC
GG
SEQ ID NO: 29 q29
AGGGGAATAACCCATTTTTTCTTGCAACCAGAATGTGGTTGCCTATACTG
GG
SEQ ID NO: 30 q4
GTCTCTGCGGAGGAAGACACTTCGGCTTCGCGGAATCGACTATCAGGCGG
GG
SEQ ID NO: 31 q6
GACTTATCCCCCAAGGATTTGTACGTGACTCCTTATAAGGTATGTCGTGC
GG
SEQ ID NO: 32 q8
TTCGTCTATTCCTGGTGGACAGTTATAAGTTCTCGACCCATTGACGCCTT
GG
SEQ ID NO: 33 q37
TGATGGCGGTTCAGGCACACAGGGGTCCGTGCGGTTCCGCACAGGGCAAT
GG
SEQ ID NOs 34-60 synthetic binding tags:
AWSHPQFEKGGGSGGGSGGSSAWSHPQFEK
(SEQ ID NO: 34)
APPGHHHWHIHH
(SEQ ID NO: 35)
MSASSYASFSWS
(SEQ ID NO: 36)
KPSHHHHHTGAN
(SEQ ID NO: 37)
MSPHPHPRHHHT
(SEQ ID NO: 38)
MSPHHMHHSHGH
(SEQ ID NO: 39)
LPHHHHLHTKLP
(SEQ ID NO: 40)
APHHHHPHHLSR
(SEQ ID NO: 41)
RGRRRRLSCRLL
(SEQ ID NO: 42)
HPPMNASHPHMH
(SEQ ID NO: 43)
HTKHSHTSPPPL
(SEQ ID NO: 44)
CHKKPSKSC
(SEQ ID NO: 45)
CTSPHTRAC
(SEQ ID NO: 46)
CSYHRMATC
(SEQ ID NO: 47)
RLNPPSQMDPPF
(SEQ ID NO: 48)
QTWPPPLWFSTS
(SEQ ID NO: 49)
YITPYAHLRGGN
(SEQ ID NO: 50)
KSLSRHDHIHHH
(SEQ ID NO: 51)
LDHSLHS
(SEQ ID NO: 52)
MHRSDLMSAAVR
(SEQ ID NO: 53)
KLPGWSG
(SEQ ID NO: 54)
AFILPTG
(SEQ ID NO: 55)
LSNNNLR
(SEQ ID NO: 56)
AAPSHEHRHSRQ
(SEQ ID NO: 57)
ALAHNPKTTHHR
(SEQ ID NO: 58)
ERPLHIHYHKGQ
(SEQ ID NO: 59)
TTHSKHHFPSSA
(SEQ ID NO: 60

1: A method of removing at least one uracil containing polynucleotide from a composition containing at least one polynucleotide, the method comprising the steps of:
(a) Providing a composition containing at least one polynucleotide;
(b) Contacting the composition with at least one uracil DNA glycosylase enzyme which is capable of forming a stable bond with a uracil containing polynucleotide, to form one or more enzyme-uracil containing polynucleotide complexes; and
(c) Separating the one or more enzyme-uracil containing polynucleotide complexes from the composition.

2: A method of blocking at least one uracil containing polynucleotide within a composition containing at least one polynucleotide, the method comprising:
(a) Providing a composition containing at least one polynucleotide; and
(b) Contacting the composition with at least one uracil DNA glycosylase enzyme which is capable of forming a stable bond with a uracil containing polynucleotide, to form one or more enzyme-uracil containing polynucleotide complexes.

3.-4. (canceled)

5: The method of claim 1, wherein the uracil DNA glycosylase enzyme is capable of forming a covalent or ionic bond with the uracil containing polynucleotide.

6: The method of claim 1, wherein the uracil DNA glycosylase enzyme is a UDGx enzyme.

7: The method of claim 1, wherein the uracil DNA glycosylase enzyme comprises an amino acid sequence having at least 70% identity with a sequence according to any of SEQ ID NOs: 1-12.

8: The method of claim 1, wherein the uracil DNA glycosylase enzyme comprises an amino acid sequence having at least 70% identity with a sequence according to SEQ ID NO: 2, 7, or 9, and wherein the amino acid sequence comprises one or more ancestral substitution mutations.

9: The method of claim 1, wherein the uracil DNA glycosylase enzyme comprises an amino acid sequence according to SEQ ID NO: 6, 8 or 10.

10: The method of claim 1, wherein the uracil DNA glycosylase enzyme is attached to a binding molecule.

11: The method of claim 10, wherein the binding molecule is selected from the group consisting any of: a silica binding tag, a biotin tag, a streptactin/Strep tag, a His-tag, a streptavidin tag, a cellulose binding domain, a MBP, and a GST.

12: The method of claim 10, wherein the separating step (c) comprises contacting the composition with one or more capture partners, which are capable of binding to the binding molecule, and separating one or more captured binding molecule-enzyme-uracil containing polynucleotide complexes from the composition.

13: The method of claim 12, wherein the capture partner is selected from the group consisting of: silica, streptactin, streptavidin, Ni-NTA, avidin, cellulose, maltose, and glutathione.

14: The method of claim 1, wherein the composition contains a plurality of polynucleotides.

15: The method of claim 1, wherein the polynucleotide is single stranded DNA.

16: The method of claim 1, wherein step (a) comprises synthesizing a composition containing at least one polynucleotide by enzymatic polynucleotide synthesis.

17: The method of claim 1, wherein the method results in a C to U conversion.

18: The method of claim 1, wherein the uracil DNA glycosylase enzyme is a modified, variant or mutant uracil DNA glycosylase enzyme.

19: The method of claim 18, wherein the uracil DNA glycosylase enzyme comprises one or more modifications, wherein the one or more modifications improve stability.

20: The method of claim 1, wherein the uracil DNA glycosylase enzyme is a UDG enzyme derived from a prokaryotic organism.


SEQUENCE LISTING (60 sequences)

SEQ ID NO: 1 - protein, 208 aa, Mycobacterium smegmatis
  REGION 1..208: note = UDGx1 from Mycobacterium smegmatis
MGAQDFVPHTADLAELAAAAGECRGCGLYRDATQAVFGAGGRSARIMMIGEQPGDKEDLAGLPFVGPAGRLLDRALEAADIDRDALYVTNAVKHFKFTRAAGGKRRIHKTPSRTEVVACRPWLIAEMTSVEPDVVVLLGATAAKALLGNDFRVTQHRGEVLHVDDVPGDPALVATVHPSSLLRGPKEERESAFAGLVDDLRVAADVRP

SEQ ID NO: 2 - protein, 215 aa, Mycobacterium thermoresistibile
  REGION 1..215: note = UDGx2 from Mycolicibacterium thermoresistibile
MAVTGAARFVPATRDLGELAEAVHACKGCDLYVDATQAVFGAGPGTAPMVMVGEQPGDREDTAGQPFVGPAGRLLQRALDAAGIDRAEVYVTNAVKHFTFTRGRRLIHKTPSRSDVVACRPWLIAELDSVRPEVVVLLGATAAKSLLGPDFRLTAHRGEVLRLPAGDATVGLGVDPLVVVTVHPSAVLRGRPGDRAEAFDALVADLEVAAGLMGS

SEQ ID NO: 3 - protein, 219 aa, Gandjariella thermophila
  REGION 1..219: note = UDGx3 from Gandjariella thermophila
MGVAQRQSAAPFVPSGAGIDELREAASGCRGCSLYRDATQTVFGQGSPRARLMMIGEQPGDREDRQGAPFVGPAGRLLNRALEEAGLPRDSVYLTNAVKHFKFERVHGKQRIHKKPSRTEVVACWPWLAAELGVVRPEFAVCLGATAAQALLGTSFRVTAHRGELLDGPRYADDADPLVVVATVHPSSVLRAPDPEAREDAYRRLVADLGVAASAMAGW

SEQ ID NO: 4 - protein, 214 aa, Rhodococcus ruber
  REGION 1..214: note = UDGx4 from Rhodococcus ruber
MAVSRQPGAEEFVPDSTDLAELAAAAGDCRGCELYRNAERTVFGAGPASARLVLVGEQPGDQEDRAGEPFVGPAGRLLDRALEEAGIDRDEVYVTNAVKHFKFERAAAGGRRIHKKPARGEIVACRPWLVAELQAVRPDVLVCLGATAAQSLLGPSFRVTAHRGEILHPDVRVPSDPAVVATIHPSAILRGPSAQREEALAGLVADLRVAAGVL

SEQ ID NO: 5 - protein, 223 aa, Homo sapiens
  REGION 1..223: note = UDGx5 from Homo sapiens
MEFFGESWKKHLSGEFGKPYFIKLMGFVAEERKHYTVYPPPHQVFTWTQMCDIKDVKVVILGQNPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTANPSPLSVYRGFFGCRHFSKTNELLQKSGKKPIDWKEL

SEQ ID NO: 6 - protein, 213 aa, synthetic construct
  REGION 1..213: note = UDGx6 modified (synthetic) 182-anc
MAVTGAARFVPETRDLGELAEAAHGCKGCDLYRDATQTVFGAGPGSAPMMLVGEQPGDQEDRAGQPFVGPAGRLLDRALEEAGIDRAQVYVTNAVKHFKFTRGKRRIHKTPSRTEVVACRPWLIAELDSVRPEVVVCLGATAAQSLLGPDFRVTAHRGEVLRLPAEDATVTVDVDPRVVVTVHPSAVLRGRPEDRAEAFDALVADLRVAAGLM

SEQ ID NO: 7 - protein, 222 aa, Mycobacterium colombiense
  REGION 1..222: note = UDGx7 from Mycobacterium colombiense
MAATSNAPGAARYLPEERGLDALRDAAETCHGCSLFEDATQTVFGNGHPGAPIMLVGEQPGDQEDRAGEPFVGPAGRLLDRALEDAGIDPAMVYETNAVKHFKFTRKGGKRRIHQKPGRTEVVACRPWLIAEIEAVRPQVIVCLGATAAQSLLGATFRVSTQRGQQLRLPSTVDVHLTPEPTLVATVHPSSVLRDRSDRHDEVYEAFVDDLRSAGAGLGRSG

SEQ ID NO: 8 - protein, 218 aa, synthetic construct
  REGION 1..218: note = UDGx7 modified (synthetic) 6-anc
MAATSNAPGADRYLPEERDLDALRDAAETCRGCSLFEDATQTVFGNGHPGAPIMLVGEQPGDQEDRAGEPFVGPAGRLLDRALEDAGIDPALVYVTNAVKHFKFTRKGGKRRIHQKPGRTEVVACRPWLIAEIEAVRPEVIVCLGATAAQSLLGSDFRVSAQRGQQLRLPASVDVDLAPEPTVVATVHPSSVLRDRSDRHDEAYQSFVDDLRSAGGGL

SEQ ID NO: 9 - protein, 214 aa, Rhodococcus rhodochrous
  REGION 1..214: note = UDGx8 from Rhodococcus rhodochrous
MAVSRQPGAGEFVPETTDLAELAAAASGCRGCDLYRNAERTVFGAGPATARLVLVGEQPGDQEDRAGEPFVGPAGRLLDRALEEAGIDRGEVYVTNAVKHFKFERAAAGGRRIHKKPARGEVVACRPWLVAELQAVRPEVLVCLGATAAQSLLGPSFRVTAHRGEILHPDAEIPSDPAVVATIHPSAILRGPSEQREEALAGLVADLRVAAGAL

SEQ ID NO: 10 - protein, 214 aa, synthetic construct
  REGION 1..214: note = UDGx8 modified (synthetic) 179-anc
MAVSRQPGAAEFVPETRDLAELAAAARGCRGCDLYRDATQTVFGAGPATARMMLVGEQPGDQEDRAGEPFVGPAGRLLDRALEEAGIDRERVYVTNAVKHFKFTRAEGGKRRIHKKPSRTEVVACRPWLVAELQAVRPEVLVCLGATAAQSLLGPSFRVTAHRGEVLHLPAEVESDPRVVATVHPSAVLRGPSEDRDEAFAALVADLRVAAGAL

SEQ ID NO: 11 - protein, 213 aa, Amycolatopsis viridis
  REGION 1..213: note = UDGx9 from Amyloclaptosis virids
MATSTRRDAAEFVPDSRSLDRLRSAALRCQGCDLHRDATQTVFGDGPAPAKVLMLGEQPGDKEDVAGEPFVGPAGRLLDRALDEAGVDRSQVYVTNAVKHFKFVRGERGKQRIHKKPSRGEIVACRPWLVAELEAVQPSVVVLLGATAASSLMGPSFRVTAHRGELLPAPEEFAGRPERVLATVHPSSVLRAPDRDEAYAALVADLRSVPEMM

SEQ ID NO: 12 - protein, 222 aa, unidentified
  source 1..222: note = Bovine mut
  REGION 1..222: note = UDGx10 from Bovine mut
MIFGESWKKHLSGEFGKPYFIKLMGFVAEERKHYTVYPPPHQVFTWTQMCDIRDVKVVILGQNPYHGPNQAHGLCFSVQRPVPPPPSLENIYKELSTDVDGFVHPGHGDLSGWAKQGVLLLNAVLTVRAHQANSHKERGWEQFTDAVVSWLNQNAHGLVFLLWGSYAQKKGSAIDRKRHHVLQTANPSPLSVYRGFFGCRHFSKTNELLQKSGKEPINWKDL

SEQ ID NO: 13 - DNA, 15 bp, synthetic construct
  source 1..15: mol_type = other DNA
  modified_base 8: mod_base = OTHER; note = ideoxyU
  misc_feature 1: note = 56-FAM
  misc_feature 1..15: note = synthetic FAM-U-oligo
tttttttnttttttt

SEQ ID NO: 14 - DNA, 15 bp, synthetic construct
  source 1..15: mol_type = other DNA
  misc_feature 1: note = 56-TAMN
  misc_feature 1..15: note = synthetic TAM-oligo
ttttttttttttttt

SEQ ID NO: 15 - DNA, 84 bp, Homo sapiens
  source 1..84: mol_type = other DNA
  misc_feature 1..84: note = HER2
acggacgtgggatcctgcaccctcgtctgccccctgcacaaccaagaggtgacagcagaggatggaacacagcggtgtgagaag

SEQ ID NO: 16 - DNA, 84 bp, synthetic construct
  source 1..84: mol_type = other DNA
  misc_feature 1..84: note = synthetic HER2-U
  modified_base 22: mod_base = OTHER; note = ideoxyU
  modified_base 27: mod_base = OTHER; note = ideoxyU
  modified_base 32: mod_base = OTHER; note = ideoxyU
  modified_base 37: mod_base = OTHER; note = ideoxyU
acggacgtgggatcctgcaccntcgtntgccncctgnacaaccaagaggtgacagcagaggatggaacacagcggtgtgagaag

SEQ ID NO: 17 - DNA, 19 bp, synthetic construct
  source 1..19: mol_type = other DNA
  misc_feature 1..19: note = Fwd control
acggacgtgggatcctgca

SEQ ID NO: 18 - DNA, 23 bp, synthetic construct
  source 1..23: mol_type = other DNA
  misc_feature 1..23: note = Rev control
cttctcacaccgctgtgttccat

SEQ ID NO: 19 - DNA, 19 bp, synthetic construct
  source 1..19: mol_type = other DNA
  misc_feature 1..19: note = 5 prime Fwd
  modified_base 2: mod_base = OTHER; note = ideoxyU
anggacgtgggatcctgca

SEQ ID NO: 20 - DNA, 23 bp, synthetic construct
  source 1..23: mol_type = other DNA
  modified_base 21: mod_base = OTHER; note = ideoxyU
  misc_feature 1..23: note = 5 prime Rev
cttctcacaccgctgtgttcnat

SEQ ID NO: 21 - DNA, 19 bp, synthetic construct
  source 1..19: mol_type = other DNA
  modified_base 6: mod_base = OTHER; note = ideoxyU
  misc_feature 1..19: note = Center-Fwd
acggangtgggatcctgca

SEQ ID NO: 22 - DNA, 23 bp, synthetic construct
  source 1..23: mol_type = other DNA
  misc_feature 1..23: note = Center-Rev
  modified_base 11: mod_base = OTHER; note = ideoxyU
cttctcacacngctgtgttccat

SEQ ID NO: 23 - DNA, 19 bp, synthetic construct
  source 1..19: mol_type = other DNA
  modified_base 18: mod_base = OTHER; note = ideoxyU
  misc_feature 1..19: note = 3 prime Fwd
acggacgtgggatcctgna

SEQ ID NO: 24 - DNA, 23 bp, synthetic construct
  source 1..23: mol_type = other DNA
  misc_feature 1..23: note = 3 prime Rev
  modified_base 4: mod_base = OTHER; note = ideoxyU
cttntcacaccgctgtgttccat

SEQ ID NO: 25 - DNA, 59 bp, synthetic construct
  source 1..59: mol_type = other DNA
  misc_feature 1..59: note = IDTq37t
  modified_base 14: mod_base = OTHER; note = ideoxyU
  modified_base 19: mod_base = OTHER; note = ideoxyU
  modified_base 23: mod_base = OTHER; note = ideoxyU
  modified_base 27: mod_base = OTHER; note = ideoxyU
  modified_base 34: mod_base = OTHER; note = ideoxyU
  modified_base 39: mod_base = OTHER; note = ideoxyU
  modified_base 44: mod_base = OTHER; note = ideoxyU
  modified_base 47: mod_base = OTHER; note = ideoxyU
  modified_base 49: mod_base = OTHER; note = ideoxyU
  modified_base 54: mod_base = OTHER; note = ideoxyU
  modified_base 45: mod_base = OTHER; note = ideoxyU
  modified_base 35: mod_base = OTHER; note = ideoxyU
  modified_base 25: mod_base = OTHER; note = ideoxyU
ttacagatgatggnggttnaggnananaggggtnngtgnggttnngnanagggnaatgg

SEQ ID NO: 26 - DNA, 59 bp, synthetic construct
  source 1..59: mol_type = other DNA
  misc_feature 1..59: note = IDTq37
ttactagtgatggcggttcaggcacacaggggtccgtgcggttccgcacagggcaatgg

SEQ ID NO: 27 - DNA, 52 bp, synthetic construct
  source 1..52: mol_type = other DNA
  misc_feature 1..52: note = q1
acgacctacagaacaaaccggggttccgagcggtaatagcaacaccaacggg

SEQ ID NO: 28 - DNA, 52 bp, synthetic construct
  source 1..52: mol_type = other DNA
  misc_feature 1..52: note = q21
gtatggcgcgatgactcgcgcacgctacggattcacttgctaaatatcacgg

SEQ ID NO: 29 - DNA, 52 bp, synthetic construct
  source 1..52: mol_type = other DNA
  misc_feature 1..52: note = q29
aggggaataacccattttttcttgcaaccagaatgtggttgcctatactggg

SEQ ID NO: 30 - DNA, 52 bp, synthetic construct
  source 1..52: mol_type = other DNA
  misc_feature 1..52: note = q4
gtctctgcggaggaagacacttcggcttcgcggaatcgactatcaggcgggg

SEQ ID NO: 31 - DNA, 52 bp, synthetic construct
  source 1..52: mol_type = other DNA
  misc_feature 1..52: note = q6
gacttatcccccaaggatttgtacgtgactccttataaggtatgtcgtgcgg

SEQ ID NO: 32 - DNA, 52 bp, synthetic construct
  source 1..52: mol_type = other DNA
  misc_feature 1..52: note = q8
ttcgtctattcctggtggacagttataagttctcgacccattgacgccttgg

SEQ ID NO: 33 - DNA, 52 bp, synthetic construct
  source 1..52: mol_type = other DNA
  misc_feature 1..52: note = q37
tgatggcggttcaggcacacaggggtccgtgcggttccgcacagggcaatgg

SEQ ID NO: 34 - protein, 30 aa, synthetic construct
  REGION 1..30: note = Streptactin tag
AWSHPQFEKGGGSGGGSGGSSAWSHPQFEK

SEQ ID NO: 35 - protein, 12 aa, synthetic construct
  REGION 1..12: note = Synthetic binding tag
APPGHHHWHIHH

SEQ ID NO: 36 - protein, 12 aa, synthetic construct
  REGION 1..12: note = Synthetic binding tag
MSASSYASFSWS

SEQ ID NO: 37 - protein, 12 aa, synthetic construct
  REGION 1..12: note = Synthetic binding tag
KPSHHHHHTGAN

SEQ ID NO: 38 - protein, 12 aa, synthetic construct
  REGION 1..12: note = Synthetic binding tag
MSPHPHPRHHHT

SEQ ID NO: 39 - protein, 12 aa, synthetic construct
  REGION 1..12: note = Synthetic binding tag
MSPHHMHHSHGH

SEQ ID NO: 40 - protein, 12 aa, synthetic construct
  REGION 1..12: note = Synthetic binding tag
LPHHHHLHTKLP

SEQ ID NO: 41 - protein, 12 aa, synthetic construct
  REGION 1..12: note = Synthetic binding tag
APHHHHPHHLSR

SEQ ID NO: 42 - protein, 12 aa, synthetic construct
  REGION 1..12: note = Synthetic binding tag
RGRRRRLSCRLL

SEQ ID NO: 43 - protein, 12 aa, synthetic construct
  REGION 1..12: note = Synthetic binding tag
HPPMNASHPHMH

SEQ ID NO: 44 - protein, 12 aa, synthetic construct
  REGION 1..12: note = Synthetic binding tag
HTKHSHTSPPPL

SEQ ID NO: 45 - protein, 9 aa, synthetic construct
  REGION 1..9: note = Synthetic binding tag
CHKKPSKSC

SEQ ID NO: 46 - protein, 9 aa, synthetic construct
  REGION 1..9: note = Synthetic binding tag
CTSPHTRAC

SEQ ID NO: 47 - protein, 9 aa, synthetic construct
  REGION 1..9: note = Synthetic binding tag
CSYHRMATC

SEQ ID NO: 48 - protein, 12 aa, synthetic construct
  REGION 1..12: note = Synthetic binding tag
RLNPPSQMDPPF

SEQ ID NO: 49 - protein, 12 aa, synthetic construct
  REGION 1..12: note = Synthetic binding tag
QTWPPPLWFSTS

SEQ ID NO: 50 - protein, 12 aa, synthetic construct
  REGION 1..12: note = Synthetic binding tag
YITPYAHLRGGN

SEQ ID NO: 51 - protein, 12 aa, synthetic construct
  REGION 1..12: note = Synthetic binding tag
KSLSRHDHIHHH

SEQ ID NO: 52 - protein, 7 aa, synthetic construct
  REGION 1..7: note = Synthetic binding tag
LDHSLHS

SEQ ID NO: 53 - protein, 12 aa, synthetic construct
  REGION 1..12: note = Synthetic binding tag
MHRSDLMSAAVR

SEQ ID NO: 54 - protein, 7 aa, synthetic construct
  REGION 1..7: note = Synthetic binding tag
KLPGWSG

SEQ ID NO: 55 - protein, 7 aa, synthetic construct
  REGION 1..7: note = Synthetic binding tag
AFILPTG

SEQ ID NO: 56 - protein, 7 aa, synthetic construct
  REGION 1..7: note = Synthetic binding tag
LSNNNLR

SEQ ID NO: 57 - protein, 12 aa, synthetic construct
  REGION 1..12: note = Synthetic binding tag
AAPSHEHRHSRQ

SEQ ID NO: 58 - protein, 12 aa, synthetic construct
  REGION 1..12: note = Synthetic binding tag
ALAHNPKTTHHR

SEQ ID NO: 59 - protein, 12 aa, synthetic construct
  REGION 1..12: note = Synthetic binding tag
ERPLHIHYHKGQ

SEQ ID NO: 60 - protein, 12 aa, synthetic construct
  REGION 1..12: note = Synthetic binding tag
TTHSKHHFPSSA